(19) United States
(12) Patent Application Publication
(10) Pub. No.: US 2022/0111257 A1
(43) Pub. Date: Apr. 14, 2022

(54) System, Method and Computer Program Product Configured for Sensor-Based Enhancement of Physical Rehabilitation

(71) Applicant: Celloscope Ltd., Tel Aviv (IL)

(72) Inventors: Guy TSUR, Tel Aviv (IL); Shahar DAVIDSON, Rehovot (IL); Zohar Netanel BOUCHRIS, Tel Aviv (IL); Hila GLICK, Tel Aviv (IL)

(21) Appl. No.: 17/500,744

(22) Filed: Oct. 13, 2021

Related U.S. Application Data
(60) Provisional application No. 63/090,973, filed on Oct. 13, 2020.

(51) Int. Cl. A63B 24/00; A61B 5/11; A61B 5/00; G16H 20/30; G16H 10/60; G06N 3/04

(52) U.S. Cl. CPC A63B 24/0075; A61B 5/112; A61B 5/7267; A61B 5/7282; A61B 2562/0219; G16H 10/60; G06N 3/04; A61B 2505/09; G16H 20/30

(57) ABSTRACT

A sensor-driven physiotherapy system comprising an exercise repository electronically storing digitally represented exercises each of which includes exercise quality parameter/s, and/or a hardware processor configured to assign a score for exercise quality parameter/s for each of at least some exercises in the repository, wherein the score is derived from sensor data collected while patients performed the exercises wearing a sensor which generates the sensor data; and/or exercise recommendation functionality to select, from the digitally represented exercises, exercise/s which the system recommends for at least one patient profile e.g. set of patients. Typically the exercise which the system recommends, for a given patient profile, statistically contributes more, to gait improvement of patients belonging to that profile, than exercises which are not recommended for that profile contribute, to gait improvement of patients belonging to that profile.

### REFERENCE TO CO-PENDING APPLICATIONS

Priority is claimed from U.S. Provisional Patent Application No. 63/090,973 “Efficient System Configured To Facilitate Physical Rehabilitation” filed on Oct. 13, 2020, the disclosure of which application/s is hereby incorporated herein by reference.

### FIELD OF THIS DISCLOSURE

The present invention relates generally to wearable sensors, and more particularly to processing sensor data.

### BACKGROUND FOR THIS DISCLOSURE

The following link: www.jpost.com/opinion/hillels-tech-corner-tel-aviv-start-up-taking-tech-one-step-further-619140 describes that typically, “patients only meet with physical therapist . . . one or two times a week . . . . But what about the other 166 hours? . . . Patients are left . . . with no way to measure progress” and describes OneStep technology.

A co-pending patent document (aka the co-pending mobile-phone-based therapy patent document) entitled System, Method And Computer Program Product For Detecting A Mobile Phone Users Risky Medical Condition, to Naveh et al (Publication number: 20200375544, Filed: Mar. 10, 2020, Publication date: Dec. 3, 2020) describes stroke detection methods operative to detect strokes suffered by mobile communication device users, and a system comprising a hardware processor operative in conjunction with a mobile communication device having at least one built-in sensor; the hardware processor being configured to, repeatedly and without being activated by the device's user, compare data derived from the at least one sensor to at least one baseline value for at least one indicator of user well-being, stored in memory accessible to the processor and/or make a stroke risk level evaluation; and/or perform at least one action if and only if the stroke risk level is over a threshold.

A co-pending patent document (aka the co-pending gait-assessment patent document) is entitled System, Method And Computer Program Product For Assessment Of A User's Gait to Naveh et al (Publication number: 20200289027, Filed: Oct. 22, 2019, Publication date: Sep. 17, 2020) and describes a gait monitoring system operative to monitor gait of an end-user bearing a wearable device equipped with at least one magneto-inertial sensor, the system comprising a processor configured to receive raw sensor data from the wearable device's at least one magneto-inertial sensor to extract situational data from the raw sensor data, the situational data including at least the device's bodily position relative to the end-user, to determine a gait analysis process which yields at least one parameter characterizing the end-user's gait, depending at least on the device's bodily position as extracted, and to compute, and generate an output indication of, the at least one parameter characterizing the end-user's gait, by running the gait analysis process as selected.

Health is an Apple app which holds health data and can also pull such data from other devices such as trackers, smartwatches and smart scales. HealthKit is an accompanying developer application programming interface (aka API) included in the iOS Software Development Kit aka SDK for the Mac which may be used when generating software applications to have extensibility and to interact with health and fitness applications on iOS.

Detecting repeated motion patterns via_Dynamic Programming using motion density is known and is described e.g. in www.researchgate.net/publication/224557414_Detecting_repeated_motion_patterns_via_Dynamic_Programming_using_motion_density.

Padding is a known technology in the art of neural networks and is described e.g. here; machinelearningmastery.com/padding-and-stride-for-convolutional-neural-networks/.

Rotation arithmetic is known in the art of Quaternions and spatial rotation e.g. as described here: en.wikipedia.org/wiki/Quaternions_and_spatial_rotation.

The disclosures of all publications and patent documents mentioned in the specification, and of the publications and patent documents cited therein directly or indirectly, are hereby incorporated by reference other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

Motion analysis, or motion capture, which yields kinematic variables such as joint angles, is known, and is referred to for example, in the following publication: jneuroengrehab.biomedcentral.com/articles/10.1186/1743-0003-3-18 which describes a protocol including set-up of cameras, positioning markers and providing an optoelectronic system for three-dimensional kinematic motion capture. Also, arxiv.org/pdf/1907.00837.pdf describes real-time monocular RGB based 3D motion capture to derive 3D skeletal pose data including plural skeletal joint angles.

### SUMMARY OF CERTAIN EMBODIMENTS

The following terms may be construed either in accordance with any definition thereof appearing in the prior art literature or in accordance with the specification, or to include in their respective scopes, the following:

Adherence e.g. to physiotherapy, may include the number of times a patient actually did an exercise, as opposed to the recommended number of times. Adherence may rely on user-app interaction data rather than sensor data.

Compliance e.g. with physiotherapy may include the quality of patient's execution of each exercise performed. Compliance may be derived from sensor data (e.g. by gait analysis as described herein,).

Improvement or progress, e.g. after a period of treatment is a parameter which is typically measured independently and may comprise all or any subset of the following metrics, separately or combined suitably, e.g. a simple or weighted average may be computed:

Metric1. Gait quality assessment over time—may be determined by gait's symmetricity (extent to which legs' stances and step lengths are symmetrical) and/or consistency and endurance (e.g. step rate which doesn't vary over a long walk).

It is appreciated that Gait parameter extraction methods are described generally in the following co-pending patent document (patents.justia.com/patent/20200289027 e.g. with reference to operation H, and are also described herein.

Metric2. Standard tests' results over time, such as 6 min walk test (6MWT) or timed up and go e.g. as described here: www.cdc.gov/steadi/pdf/TUG_test-print.pdf.

Metric3. Subjective reports over time such as Lower Extremity Functional Scale (LEFS) score or “Oxford Knee score” (OKS, patient reported outcome measure including questions about her/his activities of daily living).

Metric4. 3D skeleton pose estimation may be derived from a video of a patient exercising in front of a computer webcam. For example, arxiv.org/pdf/1907.00837.pdf describes real-time monocular RGB based 3D motion capture to derive 3D skeletal pose data including plural skeletal joint angles.

Metric5. Therapist assessment over time, e.g. therapy session summaries which may be entered by a human end-user e.g. physiotherapist: the system may prompt him or her to summarize, after every session, typically using an ordinal scale such as deterioration, no noticeable change, moderate improvement, and significant improvement.

A therapist may (e.g. based only on sessions and/or based on all or any subset of the above metrics) determine the level of progress of a patient over a given period e.g. by rating using an ordinal scale such as, say: deterioration, no noticeable change, moderate improvement, and significant improvement.

ML may be used to learn such human evaluations, as the system collects data. An exercise's contribution or benefit or efficacy is typically a characteristic of an exercise, given a specific population or sub-population or profile.

It is appreciated that the efficacy of a given exercise may be high for, say, stroke patients and simultaneously low for, say, traffic accident survivors.

Once the system has accumulated the levels of improvement of dozens or hundreds or thousands or more patients which have performed various specific exercises, the system may compute the distribution (or just central tendency e.g. mean) of improvements over all these patients, for each specific exercise. These distributions may be computed for a specific group of patients or “profile”—say, patients having a common medical and/or demographic background. The mean improvement which follows use of a given exercise E for a given profile of patients P may be used an indication of the efficacy of exercise E for patients within profile P.

The system herein typically has a user interface (perhaps more than one, e.g. one for therapist end-users and/or another for therapists' patient end-users and/or another for laymen end-users who do not have a therapist. This user interface may be configured to provide, for any profile of patients, a list of exercises, in descending order of efficacy, for a given profile P.

Exercise quality: Any suitable technology may be used to measure sensed exercise quality. Typically, each exercise has, stored in system memory, its own exercise quality criteria, as well as other stored data regarding that exercise, such as a video of the exercise being performed properly, textual and/or oral instructions on how to perform the exercise, and recommended “dosage” (how many times each exercise should be performed). For example, the exercise quality criteria for squats may include number of repetitions actually performed, range of the hip/knee angles, and rhythm or rate parameters (how long it takes to get down/up, stay down/up). Exercise quality criteria may be measured through motion analysis of phone measurements sensed by a phone (e.g. the inertial sensor thereof) which is worn by the patient e.g. in her or his pants pocket.

It is appreciated that 3D skeleton pose estimation derived from a video of a patient exercising in front of a computer webcam may contribute other parameters which may serve as exercise quality criteria.

The term PT is used herein to refer to physiotherapists and to physiotherapy.

New smart devices with advanced sensing abilities, alongside the growing demand for home healthcare and health monitoring solutions, highly affect the digital health market and specifically the use of smartphone apps that may monitor the patient's state, adherence and compliance to the treatment. With the right tools, the therapist may use this information to better suit the treatment for the patient's current state, and increase the efficacy of the treatment.

Certain embodiments seek to provide an efficient system configured to facilitate physical rehabilitation.

Certain embodiments seek to provide a personal improvement app e.g. for physiotherapy or rehabilitation e.g. gait rehabilitation.

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

Certain embodiments seek to provide a self-help e.g. rehab app, which collects patient data and schedules meetings with a human expert e.g. PT, and serves relevant portions of collected patient data to each PT before each meeting, thereby allowing each PT to effectively prepare for the meeting.

Certain embodiments seek to provide a self-help e.g. rehab app which has a bank of prompts, and includes a GUI allowing each PT to record each prompt in her or his own voice, and then presents all prompts, according to system logic, to each patient, in the voice of the PT assigned to that patient.

Certain embodiments seek to provide a self-help e.g. rehab app which guides each end-user through a cycle of operations including scheduling meetings with a human expert e.g. PT with a frequency which differs from one end-user to another, and wherein the frequency may be determined by human experts and/or may be an automatic capability which is learned from the human experts which may be corroborated by the human experts.

Certain embodiments seek to provide a self-help e.g. rehab app which tracks patient data, and each time the system tracks a major change/anomaly (i.e. step rate has changed by 10%) the therapist is alerted and prompted to mark whether this change is significant. This way, the system learns which parameters are significant, and assigns a score for each (parameter, dysfunction) pair.

Certain embodiments seek to provide a self-help e.g. rehab app which has internal logic determining which data is collected, the logic comprising any logic described herein; typically data collected depends on questions asked.

Certain embodiments seek to provide a self-help e.g. rehab app which guides each end-user through a cycle of operations which is repeated, unless and until defined recovery goal/s are met by the end-user.

Certain embodiments seek to provide a self-help e.g. rehab app which collects data over a window of time in which the patient is not interacting with the app, before generating a plan for the patient, then generates the plan based at least in part on the data.

Certain embodiments seek to provide a self-help e.g. rehab app which generates plans for end-users e.g. patients, including all or any subset of: providing a GUI enabling human experts to generate plans, collecting data quantifying how successful various plans were for various end-users, and using machine learning to analyze the data, and, accordingly, to generate recommendations for plans, and presenting recommendations for plans to human experts via the GUI, recording the human experts' modifications of the plans, if any, and learning, based on the modifications, how to present better recommendations for plans.

Certain embodiments seek to provide a self-help e.g. rehab app which generates plans for end-users based at least partly on self-reported data, collecting data quantifying how successful various plans were for various end-users, and using machine learning to analyze the data, and accordingly to generate recommendations for plans.

Certain embodiments seek to provide a self-help e.g. rehab app which collects data indicating an extent to which a patient has complied with a plan generated for her or him, and, optionally, modifies the plan based at least in part on the data and/or uses machine learning to learn to predict which exercises assigned to which patients are more and less likely to be complied with. It is appreciated that “exercises” are a special case of recommendations included within the patient or end-user's plan.

Certain embodiments seek to provide a self-help e.g. rehab app which collects data rating quality of execution of a plan e.g. exercises assigned to a patient, and, optionally, modifies the plan based at least in part on the data and/or uses machine learning to learn to predict which exercises assigned to which patients are likely to be performed at higher or lower quality. It is appreciated that “exercises” are a special case of recommendations included within the patient or end-user's plan.

Certain embodiments seek to provide any combination of the above embodiments wherein the app rehabilitates the patient's walking skills.

Certain embodiments seek to provide an automated physiotherapy system comprising:
a first processor configured to generate an evaluation of a patient's progress; and/or
   a second processor configured, responsive to at least one evaluation of at least one patient generated by the first processor, to generate an efficacy score of at least one exercise for at least one population of patients; and/or
   a third processor configured to provide a human user with a recommendation for the at least one profile/subpopulation of patients, responsive to a set of efficacy scores including plural efficacy scores of plural exercises respectively for the profile/subpopulation of patients.

The first processor typically receives sensor data from a sensor worn by the patient and generates the evaluation responsively. The recommendation typically includes an ordering of the plural exercises from most recommended to least recommended, responsive to the set of efficacy scores.

A subpopulation of patients may be defined in any suitable manner e.g. as a set of patients which have all undergone the same surgical procedure, e.g. knee replacement, or have all suffered the same injury e.g. an ankle injury, and/or each subpopulation may include patients who are all in the same age group, and/or each subpopulation may be defined by a human expert e.g. therapist who regards certain individuals as having common denominators e.g. because they have similar symptoms such as gait asymmetry or a limp and/or because they do or do not take regular physical exercise, whether general or of a specific type, and/or because they have similar results on tests such as, say, lung capacity, and/or subpopulations may be defined algorithmically, e.g. by defining a distance metric between known data points (e.g. what is the distance between a female patient age 60 who has a limp in her right foot, and a male patient age 50 who has a limp in his left foot), and then using suitable computational techniques such as cluster analysis to subdivide or even partition a group of patients into subgroups, which, using the distance metric, are close together, and are typically far from other groups. Also, a subpopulation may be a logical combination of any of the above subpopulations, such as the intersection of patients who are female, age 60-70, and have a limp in one leg.

The human user may be a therapist caring for the patient, and/or may be the patient herself or himself.

Certain embodiments seek to provide a system that derives all or any subset of the following from sensors, typically worn by a patent: the adherence, the compliance (the quality of the patient's exercising), and the quality of exercise (the exercise's contribution to the patient's progress) of a patient and an exercise.

Certain embodiments seek to provide a system that derives all or any subset of the following from sensors, typically worn by a patient: the adherence, the compliance (the quality of the patient's exercising), and the quality of exercise (the exercise's contribution to the patient's progress) of an exercise among a group of patients, and, typically, yields profiling of exercises which in turn may enable the system to operate as a recommendation system which matches or recommends at least one exercise to or for least one patient. The measurement of adherence may require the user-app interaction data, and may not require sensor data. The measurement of compliance may require sensor data (such as gait analysis, and/or other cases of analysis). Measurement of quality of a plan/exercise typically requires the evaluation of the patient's progress.

Certain embodiments seek to provide a system that teams how to measure the recovery progress/trajectory of a patient, based on gait assessment fed from smartphone sensors and a therapist's annotations. Typically, the gait assessment requires sensor data. The therapist's annotations serve to evaluate the different gait parameters into one personal measure of progress.

Certain embodiments seek to provide a system that learns the profile of a patient to adjust his plan when he does not provide or provides partial data intentionally. The data the system uses for this task may include unconscious gait and motion analysis derived from sensor data, and/or environmental sensor data such as GPS or a light sensor. The system may compare unconscious data over patients, and may then learn from patients that have more information and therapy history, and derive recommendations also for patients who have less information and therapy history.

Certain embodiments seek to provide a system that recommends plans/exercises to patients, including exercises which patients are likely to perform at higher quality, and not including exercises which patients are likely to perform at lower quality. The system may use sensor data (such as motion data) collected during exercise to assess quality of the performance of the exercise.

Certain embodiments seek to provide a system configured to provide evaluation and/or patient profiling and/or recommendation of treatment, typically based on ongoing objective measurement and analysis fed by sensor data, and more data/info fed by the processes of the treatment e.g. all or any subset of therapist feedback, comments, and annotations, and subjective reports of patients. According to one embodiment, a (typically continuous) process is provided that collects data on and from the patient and/or feeds the data to a recommendation system and/or sends the recommendation system output to a PT or other expert end-user, who uses the output to adjust the recovery plan for the patient, and then, typically, repeats. Typically, this process measures and assesses the patient's condition, adherence, compliance and improvement alongside the patient's recovery plan, and yields a set of parameters in order to optimize the patient recovery plan, targeted for TelePT. These suggestions for patient recovery plan adjustments include all or any subset of change in the exercises that the patient is asked to perform at home, their length, the time of the day, the main focus points that the patient needs to pay attention to while exercising, and more. The process might also ask the patient or the therapist for more information, which may include all or any subset of patient's subjective reports, standardized tests, or other clinical information (e.g. joint range of motion, pain level, ability to perform household tasks).

The process takes the patient from his first meeting with the service, to the state of daily use of the service, and provides valuable information to the PT, which allows him to build not only the best clinical recovery plan, but also a plan that the patient has a higher chance of following through. For example, the process may provide the PT with a set of activities that the patient does every day, e.g. walk to work, or to the car in the parking lot. This daily walk may become part of the daily exercise, and by showing that to the PT he may adjust his exercise plan using this new information, for example, asking the patient to make this walk with/without his cane, or to record himself every day on the same route, in order to see a clearer view of his ability to walk.

This is typically a continuous process, as new data keeps on coming from the patient along with changes to his condition and needs. It enhances the same protocol used in “classic PT”, where there is an evaluation meeting every week in which the PT adjusts the recovery plan.

With adherence sometimes being an important limiting factor in PT, in certain embodiments the system is operative to proactively build a habit of use, increase adherence, and to facilitate better/faster recovery. The system is also configured to conveniently monitor progress and smartly allocate resources. Typically, this is an on-going process, including a repeated sequence of operations e.g. all or any subset of the following operations (in any suitable order e.g. as per the order below):
1. On-boarding: e.g. loading the app, sign-in, providing information and goals
2. Calibration of gait and showing genuine interest by completing tasks (like walks)
3. In-person virtual meeting with a PT that builds a recovery plan that includes creating personalized exercises, adjusting the number of sets and repetitions, deciding on goals for the next time period (e.g. improving the step rate on a morning walk from 65 steps per minute to 70 steps per minute, or being able to close a bra with one hand). This plan is not only the best from the predicted clinical outcome, if adhered to, but also may be best tailored to the patient's lifestyle (for example if the patient's daily routine includes a morning walk to the park, this may be a part of his exercise plan) for the patient, based on the patient condition and goals (operation 2) and what the system has learned from Operation 3.
4. The app on the patient's phone is always aware of the plan that the patient needs to do. The app may remind the patient to do his plan at the right time for him. These reminders may be via phone notifications. After the patient opens the app, he may see a list of his exercises for the daily exercise session. He may go over them one by one, and do them with the phone on him so the app may be able to measure his movement, and provide vital feedback on the level of execution (for example, if the exercise was a squat, then it measures the range of motion, the amount of time spent in the bending position, repetitions, and how all of these compare to the goals set by the PT). For some of the exercises, the patient may be asked to add some of the information manually (e.g. number of repetitions). At the end of the exercise the patient might also be asked to provide feedback on how this exercise was, and whether it was difficult, or caused any pain, etc.
5. Back to operation 3, unless recovery goals are met, in which case, end.

In the background, the system may be configured for providing (automatically) the patients with motivational and informational notifications: “you are doing great today”, “you have completed 80% of you daily program”, “walking 5 times per week is proven to accelerate recovery”, “you got the weekly badge”, etc.

According to certain embodiments, the system stores a definition of exercise quality, for each exercise. These definitions may be generated by human experts e.g. physiotherapists. For example, the quality of a squat exercise may be defined as some function, such as a simple or weighted average, which combines the degree or angle of squatting achieved (in radians or degrees) in each of the mandated number of repetitions. The system may segment the data recorded by a sensor e.g. IMU in the subject's pocket as he exercised into repetitions, and may determine the degree or angle of squatting achieved (in radians or degrees) during each repetition performed, by analyzing the sensor data for each repetition. Or, the quality of a squat exercise may be defined as the number of repetitions performed, in which the degree or angle of squatting achieved was at least 1 radian. Similarly, the quality of an arm-raising exercise may be defined as some function, such as a simple or weighted average, which combines the angle at which the arm was raised, in each of several repetitions.

It is appreciated that system functionalities described herein as having been machine learned, may instead be achieved analytically e.g. by computation. Conversely, system functionalities described herein may be machine learned, even where the description herein indicates that the functionality is achieved analytically e.g. by computation. Machine learning may utilize training data and/or test data supplied by human experts e.g. physiotherapists who manually perform the functionality/ies to be learned.

It is appreciated that sensor data referred to herein may be data recorded by an IMU of a cellphone or other wearable communication device, and may alternatively be other types of sensors e.g. a video camera. The system may also have information about which exercise is being performed e.g. because the system knows where the user is within the app (which web page), and the system also stores knowledge stipulating which web pages within the app correspond to which exercises.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by server A. Analogously, the remote processor P may not, itself, perform all of the operations, and, instead, the remote processor P itself may receive output/s of portion/s of the operations from yet another processor/s P′, may be deployed off-shore relative to P, or “on a cloud”, and so forth.

The present invention thus typically includes at least the following embodiments:

Embodiment 1. A sensor-driven physiotherapy system comprising:
an exercise repository electronically storing digitally represented exercises each of which includes at least one exercise quality parameter;
a hardware processor configured to assign a score for at least one exercise quality parameter for each of at least some exercises in the repository of exercises, wherein the score is derived from sensor data collected while patients performed the exercises wearing a sensor which generates the sensor data: and
exercise recommendation functionality configured to select, from the digitally represented exercises, at least one exercise which the system recommends for at least one profile of patient, wherein the exercise which the system recommends, for a given profile of patient, statistically contributes more, to gait improvement of patients belonging to the given profile, than exercises which are not recommended for the given profile contribute, to gait improvement of patients belonging to the given profile.

Embodiment 2. A system according to any of the preceding embodiments wherein the hardware processor includes gait parameter estimation logic which estimates at least one gait parameter from sensor data.

Embodiment 3. A system according to any of the preceding embodiments wherein the at least one exercise, which the system recommends for at least one profile of patient, includes first and second exercises, wherein the first exercise statistically contributes more to gait improvement, of patients belonging to the given profile, than the second exercise, and wherein the first exercise is displayed to end-users of the system before the second exercise is displayed.

Embodiment 4. A system according to any of the preceding embodiments wherein the hardware processor's ability to derive the score from the sensor data is machine learned.

Embodiment 5. A system according to any of the preceding embodiments wherein the system also comprises an app used by patients during physiotherapy exercise sessions, and wherein at least one individual patient's gait is estimated by analyzing sensor data collected at times when the individual patient is not interacting with the app.

Embodiment 6. A system according to any of the preceding embodiments wherein the at least one gait parameter is estimated by the hardware processor, from sensor data pertaining to only one single gait cycle having a gait cycle duration.

Embodiment 7. A system according to any of the preceding embodiments wherein the at least one gait parameter is estimated by extraction of predefined gait events for each leg and determining proportions e.g. percentages of the gait cycle duration which are occupied by the predefined gait events respectively.

Embodiment 8. A system according to any of the preceding embodiments wherein the gait events include at least one of heel-strike for right leg, heel-strike for left leg, toe off for right leg, and toe-off for left leg.

Embodiment 9. A system according to any of the preceding embodiments wherein the extraction is machine-learned.

Embodiment 10. A system according to any of the preceding embodiments wherein the extraction which is machine-learned receives a repetitive motion representation, comprising a standard representation of a repetitive motion identified in the sensor data, and uses a temporal machine-learning model to produce indices respectively corresponding to the gait events.

Embodiment 11. A system according to any of the preceding embodiments wherein the standard representation of the repetitive motion is fed to a classifier which classifies which activity is being performed by the patient.

Embodiment 12. A system according to any of the preceding embodiments wherein the standard representation of the repetitive motion is fed to a classifier which classifies the bodily position at which the sensor is deployed.

Embodiment 13. A system according to any of the preceding embodiments wherein, to identify the repetitive motion in the sensor data, the sensor data is standardized by providing continuity of measurements generated by the sensor.

Embodiment 14. A system according to any of the preceding embodiments wherein, to identify the repetitive motion in the sensor data, the sensor data is standardized by providing consistency of intervals between measurements generated by the sensor.

Embodiment 15. A system according to any of the preceding embodiments wherein the repetitive motion representation typically has 6 components e.g. including yaw and/or pitch and/or roll components of a patient's gait's rotation and x and/or y and/or z components of a patient's gait's acceleration where x is the patient's gait's direction of movement and wherein the extraction typically uses a trained neural network whose input layer may comprise 6 L neurons and whose last layer may comprise GL neurons where G is a number of gait events to be estimated and wherein L is a desired number of temporal subdivisions of the repetitive motion's duration thereby to yield L time-units and wherein distributions of each of the G events' probabilities to occur in each time unit/index may be provided.

Embodiment 16. A system according to any of the preceding embodiments and wherein the trained neural network has inner layers including a cyclic convolutional layer.

Embodiment 17. A system according to any of the preceding embodiments wherein sensor data is also collected at times other than during physiotherapy exercise sessions and is used by the hardware processor to estimate at least one gait parameter G at times other than during physiotherapy exercise sessions and wherein the gait parameter G is used to quantify the gait improvement.

Embodiment 18. A system according to any of the preceding embodiments wherein the at least one gait parameter include at least one of: step length, stride length, stride width.

Embodiment 19. A system according to any of the preceding embodiments wherein the at least one gait parameter includes at least one temporal clinical standard parameter.

Embodiment 20. A system according to any of the preceding embodiments wherein the at least one gait parameter includes at least one spatial clinical standard parameter.

Embodiment 21. A system according to any of the preceding embodiments wherein when the extraction is machine-learned, and machine learning is performed plural times for each of plural bodily positions in which the sensor may be deployed, thereby to yield plural machine learning models trained against plural respective ground truth datasets.

Embodiment 22. A system according to any of the preceding embodiments wherein the hardware processor is configured to estimate improvement of a patient's gait, by deriving the improvement from the at least one gait parameter extracted from the sensor data.

Embodiment 23. A system according to any of the preceding embodiments wherein the sensor comprises an IMU.

Embodiment 24. A system according to any of the preceding embodiments which provides an output indication of body joint kinematics for at least one joint of at least one patient, thereby to present a visualization of the patient's gait.

Embodiment 25. A system according to any of the preceding embodiments wherein the exercise quality parameters include at least one of the following non-machine learned exercise quality parameters: a number of repetitions performed by the patient, a cadence parameter, and an indication of a change of the patient's orientation.

Embodiment 26. A computer program product, comprising a non-transitory tangible computer readable medium having computer readable program code embodied therein, the computer readable program code adapted to be executed to implement a sensor-driven physiotherapy method comprising:
storing digitally represented exercises each of which includes at least one exercise quality parameter, thereby to provide an exercise repository;
using a hardware processor to assign a score at least one exercise quality parameter for each of at least some exercises in the repository of exercises, wherein the score is derived from sensor data collected while patients performed the exercises wearing a sensor which generates the sensor data; and
selecting, from the digitally represented exercises, at least one exercise which the system recommends for at least one profile of patient, wherein the exercise which the system recommends, for a given profile of patient, statistically contributes more to gait improvement of patients belonging to the given profile, than exercises which are not recommended for the given profile contribute, to gait improvement of patients belonging to the given profile.

Embodiment 27. A sensor-driven physiotherapy method comprising:
storing digitally represented exercises each of which includes at least one exercise quality parameter, thereby to provide an exercise repository;
using a hardware processor to assign a score at least one exercise quality parameter for each of at least some exercises in the repository of exercises, wherein the score is derived from sensor data collected while patients performed the exercises wearing a sensor which generates the sensor data; and
selecting, from the digitally represented exercises, at least one exercise which the system recommends for at least one profile of patient, wherein the exercise which the system recommends, for a given profile of patient, statistically contributes more to gait improvement of patients belonging to the given profile, than exercises which are not recommended for the given profile contribute, to gait improvement of patients belonging to the given profile.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities e.g. within the computing system's registers and/or memories, and/or may be provided on-the-fly, into other data which may be similarly represented as physical quantities within the computing system's memories, registers or other such information storage, transmission or display devices, or may be provided to external factors e.g. via a suitable data network. The term “computer” should be broadly construed to cover any kind of electronic device with data processing capabilities, including, by way of non-limiting example, personal computers, servers, embedded cores, computing system, communication devices, processors (e.g. digital signal processor (DSP), microcontrollers, field programmable gate array (FPGA), application specific integrated circuit (ASIC), etc.) and other electronic computing devices. Any reference to a computer, controller or processor is intended to include one or more hardware devices e.g. chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably, e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface or other system illustrated or described herein. Any suitable computerized data storage e.g. computer memory may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein which may for example include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus the term user interface or “U” as used herein includes also the underlying logic which controls the data presented to the user e.g. by the system display and receives and processes and/or provides to other modules herein, data entered by a user e.g. using her or his workstation/device.

### BRIEF DESCRIPTION OF THE DRAWINGS

Example embodiments are illustrated in the various drawings. Certain embodiments of the present invention are illustrated in the following drawings; in the block diagrams, arrows between modules may be implemented as APIs, and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML.

Specifically:

FIG. 1 graphs an example motion record of an IMU measurement. This measurement was taken from a walking person. The continuous line shows the standardized data, whereas the dots show the original raw data produced by the sensors.

FIG. 2 graphs the same motion record as shown in FIG. 1 with segmentations of consecutive strides.

FIG. 3 graphs a 6-channel representation of a repetitive motion pattern. The 3 upper graphs show the 3 angular channels, their names having been inspired by the example of a walking man, while the motion is measured from the pants front pocket. Since the motion reflects the movement of the thigh, the first angle graph approximately represents the flexion and the extension of the hip joint, the second corresponds to the abduction movement, and the third one approximately shows the total rotation of the thigh and the body. The 3 lower graphs show 3 displacement channels of which the first corresponds to the movement forward, the second is the vertical displacement, and the third is the horizontal displacement.

FIGS. 4 and 5 graph flexion and acceleration amplitude extracted from IMU data. The flexion is the first of the 3 orientational (aka angular) channels produced by the process described herein with reference to FIGS. 1-3 where the calibration rotation is applied over the entire IMU data, rather than the individual pattern repetitions only. The activity recorded here shows a patient performing a timed up and go test including the forward portion thereof (FIG. 4) and the “return” portion thereof (FIG. 5).

FIG. 6 graphs flexion and acceleration amplitude extracted from IMU data. The flexion is the first of the 3 orientational channels produced by the process described herein with reference to FIGS. 1-3 where the calibration rotation is applied over the entire IMU data, rather than the individual pattern repetitions only. The activity recorded here shows a user performing a stability exercise while standing on one leg. The measurement is taken from the pants pocket of the leg raised in the air.

FIGS. 7-12 are simplified pictorial illustrations of an example GUI, wherein all or any subset of the illustrated elements may be provided e.g. as a dashboard for PTs or other human experts, and/or for patient end-users.

FIG. 13 is a simplified block diagram illustration of a physiotherapy support system which may be used standalone or in conjunction with any of the embodiments shown or described herein. All or any subset of the illustrated modules may be provided, suitably interconnected e.g. for data communication, e.g. as shown.

Methods and systems included in the scope of the present invention may include any subset or all of the functional blocks shown in the specifically illustrated implementations by way of example, in any suitable order e.g. as shown. Flows may include all or any subset of the illustrated operations, suitably ordered e.g. as shown. Tables herein may include all or any subset of the fields and/or records and/or cells and/or rows and/or columns described.

Computational, functional or logical components described and illustrated herein can be implemented in various forms, for example as hardware circuits, such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences, such as but not limited to objects, procedures, functions, routines and programs, and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology), or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer, or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if and as appropriate, and which may employ any suitable architectural option such as but not limited to FPGA, ASIC or DSP, or any suitable combination thereof.

Any hardware component mentioned herein may in fact include either one or more hardware devices e.g. chips, which may be co-located, or remote from one another.

Any method described herein is intended to include, within the scope of the embodiments of the present invention, also any software or computer program performing all or any subset of the method's operations, including a mobile application, platform or operating system e.g. as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use, and which is based on any suitable technologies such as semiconductor, magnetic, optical, paper and others.

### DETAILED DESCRIPTION OF CERTAIN EMBODIMENTS

Smartphones and other smart devices (tablets, wearables etc.) may be equipped with a wide variety of “wearable” sensors.

According to certain embodiments, an app is provided which, e.g. When triggered, uses all or any subset of the following wearable sensors: the 3-axis gyroscopes, 3 axis accelerometer, GPS, WiFi, a light sensor and a step counter, to analyze the patient's gait while walking, and adherence and compliance to the exercise plan (for example climbing stairs or doing squats). The app may be triggered manually by the patient “telling” the app “I am walking now”, or the app may be triggered automatically when the phone is moving (meaning the patient is also moving). The data from the sensors may provide insight on the patient's condition and medical state, such as a detailed analysis of his gait, how well he did (or did not do) his PT exercise plan, and, by looking at this data over time, it may give the PT a real view of the progress the patient is making (or indicate any deterioration), and possibly the reasons for such. For example, if a patient's adherence went from 5 times a week to 2 times a week, and at the same time the a-symmetry, showing the difference in stances between the legs also went up, this would be a bad sign for the overall balance of the patient. If a patient is in a recovery process (after suffering from a stroke, for example, or undergoing knee/hip replacement surgery), the process may use this data to better assess his condition and tailor a plan of exercises, treatments and more, that may help the patient to expedite his recovery, while providing clarity on his stage in the process, and what he may expect next.

Tailoring the right plan is non-trivial because the patient's progress may be a function, e.g., say, product of the clinical benefit or contribution of the plan and the patient's adherence thereto:

(Clinical benefit from plan)*(patient's adherence)*(patient's compliance)=patients progress

The patient's adherence and compliance may be affected by the plan. For example, if the plan is too hard, or takes too long, the patient might give up, will not follow the plan and will not make any progress. So, a shorter plan with less clinical benefits may actually yield better progress for the patient.

Typically the same applies to compliance, the quality of execution, as these PT exercises may not contribute to progress/improvement, if they are performed in the wrong way, and may even cause short term and long term damage. For example, after a total hip replacement surgery, the patient must exercise his hip, but taking the range of motion over the limit may impair his progress.

The process may take into account all or any of the data regarding the patient's adherence and compliance, such as percentage of exercises executed, hours of exercise, length of exercise, quality of the execution (range of motion, repeats, total time), quality of life before and after exercise (i.e. improvement in gait parameters, feeling, pain, etc.), and so on.

The process may utilize the fact that a patient is in constant interaction with his phone, e.g. in 2 ways:
I. Always on monitor—Nowadays, when a recovery plan is made for a patient, it only takes into consideration the data that can be gathered within the clinic's four walls and Q&A with the patient about what is happening outside those walls (and this subjective data from the patient might be biased, as he tries to please the PT, or present a better/worse situation than reality). The present process may be able to add data from the patient's daily life, such as the patient's gait when he is simply walking to the grocery store, and even compare that to his gait when he is doing his homework, and focuses on his gait (e.g. exercises assigned to them in the framework of the PT's plan). This may help the PT to better understand the patient's actual condition. This “breaks the clinic walls” in a way that the PT can finally understand how the patient is doing in his daily routine and jobs, rather than just the way the patient acts inside the clinic, or subjectively reports how he is doing.
II. Behavior analysis e.g. compliance and plan execution quality analysis—Another problem in recovery processes is patient adherence (whether the patient doing his plan) and compliance (whether the patient is doing his plan the right way). The present process may also create a recovery plan that is more personalized for the specific patient in terms of which exercises and when he is more likely to execute. For example, if the data suggests that the patient is more active in the mornings, the process may suggest scheduling a morning exercise routine, or if the patient is only performing 3 of the 7 given exercises, the process may ensure the first 3 exercises are those which are known to contribute the most, and/or suggest to give a lower number of daily exercises in order to increase the chances of patient adherence. In terms of compliance, if the patient is asked to do 10 squats of 60 degrees, and the app measured that he always does 5 squats whose angle is 60 degrees, and 5 squats whose angle is 30 degrees, the app may suggest the PT to change the exercise to 6 squats only, and once the patient is able to do 6 squats that meets the goal, update that number again to 7. Moreover, if the patient's goal in his own words is “to be able to play with my grandchildren again”, and he added them to his support group, the app may prompt a message from his grandchildren in times of difficulty or as a “reward” after a successful exercise session. This process is, typically, always monitored and constantly optimized, based on:
1. Specific patient actions (e.g. if the program changed the number of exercises from 5 to 7, but adherence dropped, it may be changed again. Or if a message sent at 10:30 am with an encouraging message led to an increase in adherence, another message may be sent at 10:30 am the next morning); and/or
2. Extrapolating from similar patients. The system herein may match the patient based on various properties, including demographic information such as age, gender, height, weight, geo location, and family status; medical information such as medical history, medical events, goals for their treatment, and medical observations and diagnosis by a clinician or a therapist; and continuous information such as adherence to the a plan or exercise over time, projected trajectories of gait analysis, and subjective reporting (LEFS—lower extremity functional scale, for instance). Generalized groups of patients may be defined by segmentation of each of those properties independently, or as combination, by clinical definition, or statistically as a result of a process to obtain similar behaviour of their members. Similar behaviour may be defined, for example, as the same level of adherence to the treatment, or as similar values of gait parameters. Moreover, identification of generalized groups of patients may be possible over some combination of properties using clustering algorithms. For example, once a similarity metric is defined over pairs of patients, spectral clustering may be applied. An example of a similarity metric is this: two patients are similar if they suffer from the same dysfunction and their age difference is smaller than 5. After attributing the patient to a group, the system may take into account the “insights”, already known for this group, and apply them to the patient. If the patient's generalized group is seen to have the best chance for adherence when the plan includes a low variety of exercises, then, an “insight” which may be presented to a PT may comprise a recommendation to use the same exercises from the previous week, rather than introducing new exercises (this may be presented to the PT on his GUI when he is building and adjusting the exercise plan for the patient).

The process typically includes all or any subset of the following operations or stages, in any suitable order e.g. as follows, and may be a never-ending process.
a. First data gathering
   i. “Getting to know you” questions such as all or any subset of the following:
      1. Patient's demographic, such as age, gender, height etc.
      2. Patient's medical information—Why is he doing certain exercises?Where are his areas of pain? How long has he been exercising?
      3. Patient's goals—where he wants to be at the end of this process.
      4. Patient's support group—Who are the people that help the patient in his recovery process? How can they be reached?
      5. Behavioral questionnaire—his exercise habits, what time he usually does them, how he feels while doing them. What he likes about them and what he hates about them, his best guess about the percentage of the exercises he performed over the last few months, preference for changing vs. constant exercises.
      6. Walking tests—The patient does a battery of walking tests (based on his medical questionnaire answers) that lets the app get a first good sample of a monitored state of the patient's gait. The tests may, for this example, include the 6 minutes walking test, 2 minutes walking tests, 10 meters test, timed up and go, and more, all of which are standard tests, well accepted by the PT community, and are used globally to assess the condition of patients.
      7. Standardized questionnaire/s—The patient does a battery of tests (based on his medical questionnaire answers) that lets the app get a first good assessment of his condition. The questionnaire may, for this example, include LEFS (lower extremity functional scale), VAS (Visual analog scale), PSFS (Patient Specific Functional Scale), KOOS (Knee Injury & Osteoarthritis Outcome), Foot & Ankle Disability Index, Oxford Knee Score and more, all of which are standard questionnaires, well accepted by the PT community, and are used globally to assess the condition of patients.
   ii. Getting to know your walk—The patient may record himself using smartphone sensors, in a series of walks where he is asked to perform different tasks e.g. all or any subset of the following:
      1. Walk at your highest step rate
      2. Walk at a slow step rate
      3. Walk with long steps
      4. Walk with short steps
      5. Walk while paying attention to the balance, and try to spend the
      same time on both legs
      6. Walk the way it causes you the least amount of pain
      7. Other walking movements
b. Continuous data gathering
   i. Patient's daily routine
      1. Waking hours, most active hours, daily routes/walks he takes, time he spent standing/sitting, driving etc.
      2. This may be gathered from the phone's pedometer, “Apple's health kit” (if available), and a smart triggering system (for instance record everything that is going on after getting a “significant motion” signal from the android OS, if the pedometer is going up, keep recording until it does not, this may provide a sample “spontaneous” walk or movement).
      3. Phone interaction—from Google or Apple “Digital wellbeing” SDK, the interactions of the patient with the phone may be obtained, and it may be deduced when he is more likely to have “phone time” that may be turned into “PT time”.
   ii. Patient's adherence to exercises
      1. How many times a week does he do his exercises? When and where is he doing them? In relation to the next PT session (e.g. he always does his exercise for 3 days after the PT session, then stops, and start again a day before his next session)
      2. Relevant data may be collected at any time the patient is performing his exercises, which may include all or any subset of:
         a. Geo location—is he exercising at home? At his office? In a nearby park?
         b. Time of day
         c. Phone movement (is it placed on a table? Holding it in his hand?)
         d. Length of the exercise
   iii. Exercise compliance—how well is he executing the exercises? For each exercise, how much time he spent doing in, and each repetition, range of motion of each exercise. Either the motion sensors of the phone, or the camera on the phone/computer/tablet may be used to assess the compliance. The used algorithms might reconstruct the human skeleton from the image, motion, or a fusion of both, or measure simpler motions that have a defined start and end (e.g. total angular change when lifting the phone, that is directly correlated to the shoulder's range of motion).
   iv. Patient's walking habits and gait parameters—what are the different gait parameters (stance, step rate, stride length, hip flexion, hip extension, ankle movements etc.) of the patient while he walks in his daily life? For example, what is his regular stance when he is not in a PT session or doing his exercises, and is there a difference between the gait patterns in the morning, compared to the afternoon or evening.
   v. Other daily activities—for example, how many, when, and how well the patient is doing daily activities may be detected, and any improvement/deterioration in comparison to the past (or to other patients in the patient's cluster) may be looked for. These activities include walking up or down stairs, sitting down, or getting up from a chair, and more. The system detects these activities using the “always on monitor”, and using trained AI models to classify and analyze the activity, e.g. in accordance with any of the embodiments shown or described in the co-pending mobile-phone-based therapy patent document.
c. Analysis of the patient's data
   i. Compared to himself—integrating the total data the system gathered, looking at trends of the gait parameters between days and time of day, trends of parameters within long walks, exercise adherence and compliance, overall activeness (active minutes, total steps per day, etc.) and comparing them to previous data of the patient to monitor his progress.
   ii. Compared to other historic patients—to optimize the recovery plan, the patient's data may be compared with past data of other patients, trying to find a “similar patient” in terms of all the data mentioned above (demographic, medical and behavioral). At this point, the aspects that worked better for the “similar patients” may be included in the recommended plan.
d. Patient parameters yield a recommended recovery plan
   i. Using the analysis of the data from the previous operation, the process finds patterns that may point to which plan the patient is likely to follow (for example, if, when he had 3 exercises that took 15 minutes, he performed them 6 times a week, but when he had 5 exercises which took 25 minutes, he only did them 2 times a week, then the recommended recovery plan may be 3 daily exercises, and not 5). These parameters may include all or a subset of:
      1. Recommended exercises
      2. Exercise length in seconds
      3. Exercise repetitions
      4. Number of daily exercises per day (e.g. may be a different number for day 1 and day 3)
      5. Length of exercise plan in days (course)—the standard is 10 days per plan.
      6. Number of weekly exercise sessions (not all patients need to exercise each day)
      7. Best time of day to exercise (and set the daily reminders for it).
      8. When in the plan to add a personalized message from the PT, or from a family member.
      9. Daily activities to monitor as part of the plan (for instance “The patient walks every day at 10 AM for 15 minutes, ask the patient to record this walk daily”)
   ii. Tests/questionnaires to better assess the patient's condition.
e. PT or other human professional typically builds a tailor made recovery plan for the patient.
   A professional PT looks at all the output of the process, via web or mobile, which includes all or any subset of:
      i. The PT accesses the data where he may see all the patient's data, number of steps taken while actively recording, and also the steps which the app recorded from the background seamlessly along with all the gait parameters for those walks, and their trends over time (within the walk and over time). Also, all of the feedback from the patient may be accessible there, e.g. the patient's notes about specific walks or exercises, and/or chat messages with the PT.
      ii. The patient's current status
         1. Patient's details—age, medical condition, messages from the patient, messages from patient support group and caregivers.
         2. Total activeness in the time period—total number and daily average of active minutes, steps, stairs climbed, exercise time.
         3. How was his gait this time period (can be weekly, bi-weekly or monthly), with all of the parameters extracted, including stance, single support, double support, hip rang, hip flexion, hip extension, hip rotation, hip adduction, hip abduction, step length, step rate, velocity, stamina (how long he can keep his normal velocity before he gets tired), and more.
         a. All of the mentioned parameters are from the walks the patient actively recorded using the app, or from the “always on monitor”, meaning walks the patient did with the phone on him (in his hand, bag, pocket).
         b. For every walk detected, the PT may “zoom in” and see the trends of the gait parameters over time (for example that the patient started with a high step rate, and then lowered his step rate after 2 minutes), or compare to other parameters (e.g. see that every time the step length goes up, the step rate goes down).
         4. The patient current exercise plan
      iii. Improvement or deterioration in patient status
         1. In every parameter in the patient status, the PT may see if there is an improvement or deterioration in that area.
      iv. About every exercise in the current exercise plan
         1. Adherence—e.g. number of times the patient did the exercise. When the patient starts his daily exercise, he turns the app on, and he may see the list of exercises the PT prescribed for him for that day. The patient then goes through them one by one, and executes them while the phone is in his pocket. This way, by the end of each day, after the patient finishes each exercise, the data is uploaded to the server, and the adherence data is saved.
         2. Compliance—for each exercise the PT defines a goal for this exercise. for example, for squats, which are one of the more common exercises, the PT may define the number of times the patient needs to repeat this exercise (say, 3 sets of 5 squats each), and the maximum bend of the hip (say, an angle of 60 degrees). As described before, the patient notifies the app that he is now doing squats, and then place the phone in his pocket. The app records all the data from the phone sensors (3 axis accelerometers and 3 axis gyroscopes). This data may be sent to the server, where the analysis is performed (the analysis may also be done locally on the phone), extracting the phone location in space at each given sample (sample rate is 50-100 Hz), and as the system knows the phone position on the body. e.g. right back pocket, the app either asks the user for this data e.g. phone position, or the app may compare the measured movement in space and using a trained model to match the pattern of a squat from right back pocket (of the possible phone positions).
         3. “Red flags” about exercises that the patient did not do at all, or did poorly.
      The professional looks at the data, and, accordingly, generates the next training plan which may then be sent to the patient.
f. operations a-e may be repeated.

Typically, this is a cyclic process, as the professional determines the next time the plan needs to be updated, where he again may get all the information from the process and may build the next plan.

It is appreciated that the term “PT” is used herein by way of example, and is intended more generally to refer to any human expert or professional.

Any method or system or operation, element or component herein may be provided in combination with any of the methods or systems or any operation, element or component described in (i) co-owned U.S. Ser. No. 16/659,832, filed 22 Oct. 2019, published under publication number 2020/0289027 and entitled “ . . . assessment of a user's gait” and/or in co-pending U.S. Ser. No. 16/814,114 to Naveh et al entitled “SYSTEM, METHOD AND COMPUTER PROGRAM PRODUCT FOR DETECTING A MOBILE PHONE USER'S RISKY MEDICAL CONDITION”, (Publication number: 20200375544, Filed: Mar. 10, 2020, Publication date: Dec. 3, 2020).

Features of the present invention, including method steps, which are described in the context of separate embodiments, may also be provided in combination in a single embodiment. Conversely, features of the invention, which are described for brevity in the context of a single embodiment or in a certain order may be provided separately or in any suitable sub-combination or in a different order.

Any or all of computerized sensors, output devices or displays, processors, data storage and networks may be used as appropriate to implement any of the methods and apparatus shown and described herein.

The invention includes but is not limited to the embodiments recited in the following claims, and to processing circuitry comprising at least one processor and at least one memory and configured to perform at least one of or any combination of the described operations or to execute any combination of the described modules.

The following is another example flow provided in accordance with certain embodiments. It is appreciated that all or any subset of operations 10, 20, . . . below and/or of their sub-operations, may be performed, in any suitable order e.g. as follows. The flow may be provided in conjunction with any of the embodiments shown and described herein.

Operation 10: For each exercise x and each patient p, the system may measure adherence e.g. using user-app interaction data but not sensor data.

For example, if the patient was instructed to use the app twice a day, for 10 days, and in practice, the patient used the app a total of 16 times rather than 20, and of the 16 times, the patient skipped the 4^{th} exercise (e.g. did not click on or otherwise select the app page which presents the 4^{th} exercise) 3 times, this means that the patient, at least according to the user-app interaction data, performed the 4^{th} exercise 16-3=13 times out of 20, yielding an adherence score of 13/20=65%.

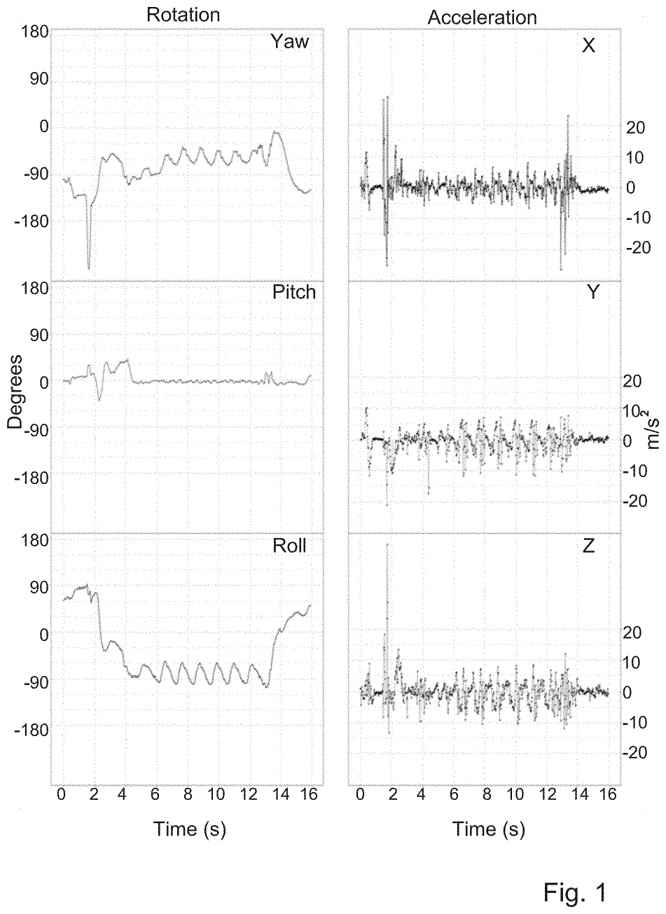

Operation 20: For each exercise x and each patient p, the system may measure compliance (=quality of patient's exercising) e.g. by estimating each exercise's criteria aka exercise quality parameters by analyzing sensor (e.g. IMU) data collected in the time-period during which patient is doing exercise x. Operation 20 may include sub-operations a, b:
a. collect IMU data in time-period during which patient is doing exercise x; and
b. segment into plural repetitive patterns, each of which typically corresponds to a single repetition of exercise x, e.g. as described herein with reference to FIGS. 1-3 or any other method for segmenting into repetitions, and then generating a standard representation of a Repetitive Pattern Measured by IMU.
c. for each of the plural repetitive patterns, each of which typically corresponds to a single repetition of exercise x, extract exercise quality parameters.

However, it is appreciated that the exercise quality parameters may be produced or extracted directly from the representation of the motion e.g. as described herein with reference to FIGS. 1-3 or any other method for segmenting into repetitions, and generating a standard representation of a Repetitive Pattern Measured by IMU.

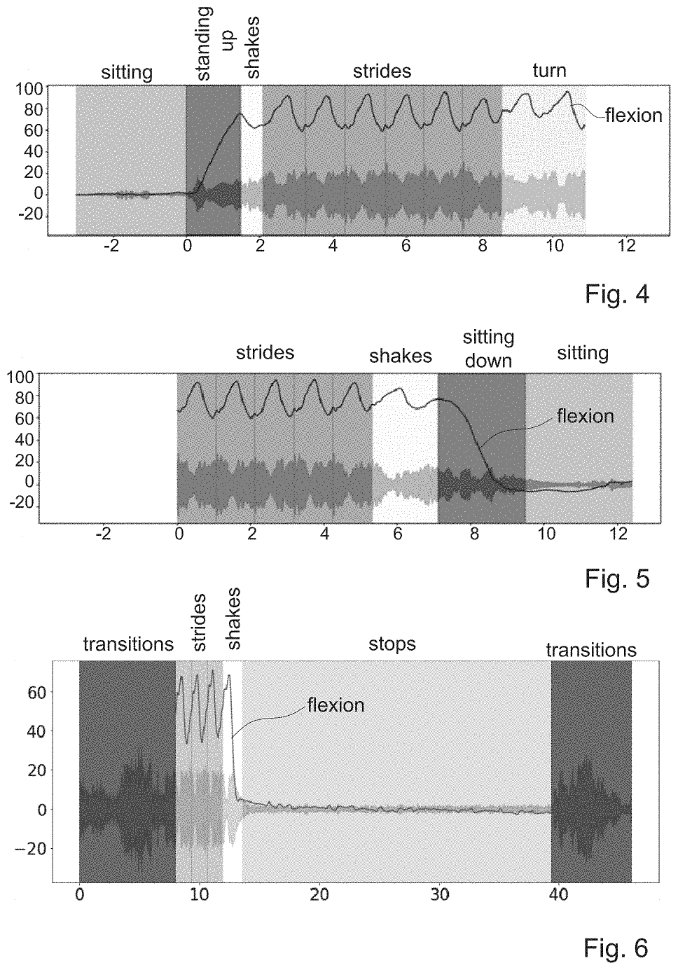

Exercise quality parameters may be extracted by a ML process e.g. as described herein with reference to FIGS. 4-6.

Example: assume compliance has plural (e.g. 2) discrete levels which score successful performance of, or quality of performance of, the exercise. These levels typically are based on quantitative exercise quality parameters and may be manually defined by an expert. For example, a successful session of squats may be defined as including only (or at least one) squats each having (or at least one of which, or 5 of which, or a majority of which, having) an angular range of 95 degrees, with a one-second stay at the bottom for at least 5 (say) repetitions.

It is appreciated that determination of certain exercise quality parameters involves determining whether a subject is in motion or motionless e.g. for equilibrium exercises, in which a patient is requested to maintain body position x, without moving (i.e. while remaining motionless) e.g. for at least y seconds. This may be done e.g. by computing an average amplitude of acceleration over a sliding window of (say) 2 seconds. Then, if the average is lower than a certain threshold value, say 0.05 (m/s\hat{ }2)\hat{ }2, this is interpreted to mean that no movement on the part of the subject has occurred.

It is appreciated that determination of certain exercise quality parameters involves determining whether a subject is/is not currently in a certain body position, such as whether a subject is/is not currently standing on one leg, e.g. for determination of quality of performance of equilibrium exercises. This may be done e.g. by identifying that the flexion of the raised leg has increased, relative to a previous point in time and/or relative to the other leg, and/or by identifying that the patient is static e.g. that the amplitude of a sliding window of IMU samples, is low, e.g. relative to the known amplitude of subjects who are walking or relative to historical data of the patient when s/he was walking.

It is appreciated that once the system has identified a patient's body position from some repetitive pattern, such as a single step or gait cycle, and once the pattern has been found, the system may determine what activity the patient is engaged in, and which position the IMU is deployed at, on the patient's body, e.g. as described in co-pending US patent application for SYSTEM, METHOD AND COMPUTER PROGRAM PRODUCT FOR ASSESSMENT OF A USER'S GAIT (Application #20200289027 issued Sep. 17, 2020)—at Operation f (“Operation f: Typically, the reconstructed motion or shale or object from operation (e), rather than the raw data, is used to identify the user's activity and device's position e.g. in user's (right left) hand vs. in (right/left/hip/shirt) pocket.”).

Operation 40: For each exercise x and each patient p, the system may, if desired, compute exercise execution (of exercise x as performed by patient p) by combining adherence of patient p to exercise x as measured in operation 10 with compliance of patient p with exercise x measured in operation 20. Typically, the function of adherence and compliance used to combine these is an increasing function vis-a-vis both adherence and compliance. For example, the system may compute:

exercise_execution (of exercise x as performed by patient p)=(Patient p's Adherence) multiplied by (Patient p's Compliance).

Operation 45: measure/compute patient p's improvement (which may be positive, negative, or zero) e.g. by collecting therapist feedback and/or by collecting a subjective report of patient and/or by gait analysis throughout the day. If improvement is computed by performing gait analysis throughout the day, operation 45 may include the following sub-operations:

Collect sensor e.g. IMU data throughout the day

Segment the sensor e.g. IMU data into plural repetitive patterns, each of which typically corresponds to a single step taken by patient, e.g. as described herein with reference to FIGS. 1-3 or any other method for segmenting into repetitions, and then generating a standard representation of a Repetitive Pattern Measured by IMU.

For each of the plural repetitive patterns, each of which typically corresponds to a single step taken by patient, perform gait analysis, e.g. using Gait Parameter extraction methods either as shown and described herein, or as known in the art.

Operation 50: use adherence and/or compliance for each exercise, provided by operations 10 and/or 20, and the improvement of the patient provided by operation 45, to derive each exercise's contribution to the patient's improvement.

For example, the system may compute contributions of each exercise_execution (for all exercises performed by patient p), to her/his improvement, e.g. using regression analysis to determine exercise_execution regression coefficient for each exercise

Typically, regression independent variables include not just data re individual exercises, but also interactions between data re individual exercises (e.g. some exercises may be more fun to do than others, so patients may do the former, thus adherence may be high relative to other exercises, however, the patients may perform the exercises badly, so compliance may be low relative to other exercises.

Any suitable set of independent variable/s may be used for each exercise, when performing regression analysis. For example, if there are 3 exercises i, ii, iii, the system may define 6 independent variables for the regression analysis—adherences for exercises i, ii, iii, and compliances for i, ii, iii, and may also define interactions between these as independent variables, e.g. some or all of: adherence to exercise i x compliance with exercise i, adherence to exercise ii x compliance with exercise ii, adherence to exercise iii x compliance with exercise iii, or even “mixed” (between exercises) interactions such as adherence to exercise ix, compliance with exercise ii. According to certain embodiments, the regression analysis may look at each exercise separately, and may define, for each exercise, each patient's adherence, compliance and (as the dependent variable) corresponding improvement.

Compliance may be a single scalar which may have a few discrete level or may be continuous. Or, e.g. if plural quality parameters or criteria are defined for a given exercise, compliance may be multivariate, or the plural quality parameters may be combined into a single compliance score e.g. by normalization and averaging of the plural quality parameters. Similarly, improvement may be a single scalar which may have a few discrete levels or may be continuous. or, e.g. if plural improvement parameters or criteria are defined for a given exercise, improvement may be multivariate, or the plural improvement parameters may be combined into a single improvement score e.g. by normalization and averaging of the plural improvement parameters.

Operation 60: Combine (e.g. average) each individual exercise's contribution to improvement, over all patients in certain profiles/subgroups, and, accordingly, generate recommendations of exercise/s for patients in certain profiles/subgroups, at least partly according to how much each exercise contributed to improvement. For example, the system may be configured for more highly recommending an exercise whose coefficient found by regression analysis is high, relative to an exercise whose coefficient found by regression analysis is low, thereby making data driven decisions on what has so far been a “gut feeling” and “experience” by PT's. For example, PTs of patients in the “post stroke, females age>60” profile may be shown a recommendation which includes the n exercises in the system which have been tried on patients in this profile, ordered such that exercises from among the n exercises which yielded a higher regression analysis coefficient are displayed before exercises from among the n exercises which yielded a lower regression analysis coefficient.

It is appreciated that the system may, according to certain embodiments, determine how many times the patient repeated each exercise in a given session. For example, the patient may have been instructed to, twice daily, perform 20 squats, whereas in practice, the patient often performed only 10 or 12 squats. This data may be collected by prompting the patient him/herself, as part of each session, to stipulate how many times he repeated the 4^{th} (say) exercise in that session, and/or this data may be collected by segmenting sensor data (e.g. IMU data) collected during the session (typically only that portion of the sensor data corresponding to the time-window during which the patient was interacting with the app-page corresponding to the 4^{th} exercise) into plural repetitive patterns (e.g. as described herein with reference to FIGS. 1-3 or any other method for segmenting into repetitions, and then generating a standard representation of a Repetitive Pattern Measured by IMU) and then counting the number of repetitive patterns.

Referring again to operation 20, according to certain embodiments, compliance is defined (for each exercise) by a human expert who may stipulate, using a system GUI, what is considered a proper performance of each exercise and/or what determines the quality of each exercise as performed by a given patient. The human expert e.g. PT typically defines exercise quality parameter/s for each exercise.

Typically, a new exercise definition session, in which a human PT defines a new exercise, includes all or any subset of the following session stages:
a. system UI for PT end-users prompts PT to enter a name for the new exercise.
b. system UI for PT end-users prompts PT to upload a video of himself/herself properly performing the new exercise.
c. system UI for PT end-users prompts PT to provide a verbal (recorded or written) description of how to properly perform the exercise.
d. system UI for PT end-users prompts PT to define at least one exercise quality parameter/s for the new exercise, typically including a name, possible scores, and verbal description of the quality of performance which earns each score.

Operation 20 may also comprise operation e, in which the system UI for PT end-users prompts PT to generate test data and training data to enable the system to machine-learn how to score each exercise quality parameter which has been defined. Typically, the PT end-user is prompted to instruct a patient to perform the new exercise (whether badly or well), and to store in memory, in association with the sensor data e.g. IMU data recorded from the patient as s/he performs the new exercise, a score for each of the exercise quality parameters which have been defined for the new exercise. The PT end-user is now prompted to repeat the same process for another patient, until the process has been completed for a predetermined number of patients (e.g. until enough test data and training data has been collected). Alternatively, no learning process is provided, and the PT end-users, in advance, i.e. before run time, simply define criteria or quality parameters, to define what is considered an exercise performed well.

Thus, and according to certain embodiments, human PT's rate (typically conventional) exercise quality parameters (such as, for a squatting exercise, depth of squat in degrees/radians) for a set of patients doing exercise x for which the system in parallel collects sensor data e.g. IMU data, thereby to provide training data and test data sets, and then machine-learning is used to teach the system, using the training data and test data sets, to assign exercise quality parameters to sensor data e.g. IMU data recorded from patients doing exercise x for which no human PT ratings are available. An embodiment which employs such ML (machine learning) parameters is described elsewhere herein e.g. with reference to FIGS. 4-6.

The video and verbal description uploaded in session stages b and c may be provided to patient end-users to which the new exercise has been assigned.

Example gait parameter extraction methods configured for extracting Gait Parameters from a single IMU Data of a Gait Cycle are now described. These are useful, for example, when seeking to determine whether a patient's gait, typically throughout his day or week, has improved, as his therapy (which typically occupies only a small portion of his day or week, such as a single hour or less) proceeds. Typically, gait analysis standard parameters are extracted from a gait cycle measured by a sensor worn by a patient or subject e.g. an inertial measurement unit (IMU) in a mobile phone which may be in the patient's pocket.

A gait cycle, being a repetitive pattern, may be represented as such e.g. as described elsewhere herein. Extraction of gait analysis standard parameters may employ a machine learning (ML) framework, and may include:
1. Collecting ground truth data for training and validation of the parameter extraction algorithms—e.g. by
   a. Simultaneous measurement of the IMU recording and external gold standard measurement systems; and
   b. Syncing the two measurement systems to provide pairs of an IMU repetition representation as input and the external measurement as labels; and
2. Outlining the general structure of such algorithms' models. Gait analysis standard parameters may include spatial parameters and temporal parameters. Example ML models for these two groups of parameters may be conventional or as described elsewhere herein.

Typically, setup of the system includes collection of ground truth and training machine learning aka ML models with (or fitting ML models to) the ground truth. Then, in real time, a repetitive motion pattern may be extracted, and exercise quality and//or gait parameters may be derived therefrom, by using the pre-trained models in real-time.

Gait parameters may include temporal and/or spatial parameters, each of which is now described.

Temporal parameters: below are several examples of temporal gait parameters:
a. Cadence [steps/min]: number of steps per minute, inferred from the cycle durations. Typically, there is no need to train an ML model for this, since cadence is available given the repetitions' durations.
b. Stance [%] (for both legs): the percentage of time the leg is supporting the body's weight, from initial contact to terminal contact.

Stance=TC−IC
c. Single support [%] (for both legs): the percentage of time one leg is the only support for the body's weight (no contralateral leg support).

Single Support=IC^{contra}−TC^{contra}

This may be termed the contralateral leg's “swing”.
d. Total double support [%]: the percentage of time both feet touch the ground.

Total Double Support=(TC−IC^{contra})+(TC^{contra}−IC)

Each of the above is defined by the following four gait events: IC—Initial Contact (Heel Strike) and TC—Terminal Contact (Toe-off) for both legs. The parameters may be defined as the percentage of the whole gait cycle duration devoted to various gait events e.g. all or any subset of the above 4 gait events. Gait cycle duration typically comprises the duration between consecutive Initial Contact events of the same leg.

The following are examples of spatial parameters:
Step length [cm] (for both legs): the distance between two consecutive heel contacts of the two legs.
Stride length [cm]: the distance between two consecutive heel contacts of the same leg.
Stride width [cm]: the distance between the center of each heel during the double support portion of a stride.
Walking speed [m/sec]: the walking velocity of the individual, which is the stride length divided by the stride duration. Given the stride length and the cadence, the walking speed may be computed directly.

ML models may be trained (conventionally, or as described elsewhere herein) to extract the four gait events defined above and/or to estimate the step length, and/or to estimate stride length, and/or to estimate stride width, thereby to yield all parameters described here.

A Ground truth data set typically comprises a multiplicity of samples of repetitive motion representation and their corresponding labels. The ground truth may serve as a representative sample of the reality, and may be derived from many, e.g. hundreds of, human subjects, who typically are selected to constitute a good representation of the general population in terms of gender, age, diagnosis, and so forth. These subjects perform various kinds of walks such as, by instruction, asymmetrical walks with different step lengths for the right and the left leg, imitations of various limitations, such as walking on the toes on one leg, or on the heels only, or not bending the knee, and so forth. The routine of walks performed by each subject may be termed the measurement protocol. Each segment or specimen of a specific kind of walk typically includes at least 20 (say) repetitions, and the movement of the patient executing each kind of walk may be measured from each of several different bodily positions occupied by the sensor (e.g. the phone on which the sensor is mounted may be in the patient's right pocket, or left pocket, or right hand, or left hand). In total, the ground truth sample may include at least hundreds of thousands of repetitive motion representations and labels, or more.

Gold standard measurement systems for gait analysis include at least the following three technologies:
a. An optoelectronic motion capture system for body kinematics collection. By placing markers on various places on the patient's body and capturing their movement on cameras, the system produces an elaborate analysis. One widely used tool in this category is the Vicon.
b. Using pressure-sensitive walkways such as Zeno and GAITRite, to collect data on a patient's gait. The walkways may be several meters long, measuring straight walking, turning, and similar maneuvers.
c. Based on wearable Inertial Measurement Units (IMUs) e.g. GaitUp and APDM, measure spontaneous walk of the subject in natural environments, using wearable systems which are cheap and accessible, whereas the first two technologies, a and b, are typically most suitable for the laboratory. In addition, some systems enable producing gait events' timestamps and precise locations during each gait cycle. For example, a pipeline may be provided producing ground truth using a pressure-sensitive treadmill system such as the Motek™ C-Mill. It is appreciated that a subject may be directed to walk on the treadmill, with an IMU (e.g. phone which includes an IMU) in one of his pockets—or 2 IMU's, one in each of her or his hip pockets. A similar pipeline may be established on other technologies as well. An example implementation may include the Motek™ C-Mill treadmill which uses pressure sensors to measure contact of each foot with the band of the treadmill, and then computes the precise time of all or any subset of the following four events during each gait cycle:
1. The initial contact of the left foot (usually the “Heel-Strike”)
2. The terminal contact of the left foot (usually the “Toe-Off”)
3. The initial contact of the right foot
4. The terminal contact of the right foot

The treadmill may also measure the precise location of the foot on the treadmill e.g. at each of the above four events, and records the speed of the treadmill at every time point.

From the location and speed information provided by the treadmill, the system may compute length and/or width of each step. For length of any given step, take the position of the foot making initial contact with the treadmill along the step's lengthwise axis (axis from heel to toe i.e. in the direction of the patient's movement as s/he took the step). Then take the position of the opposite foot at the foot's previous time of initial contact and subtract the movement of the treadmill that occurred since the previous initial contact event, from the position of the opposite foot to yield an “adjusted” position of the opposite foot. The movement of the treadmill may be computed by multiplying the speed of the treadmill by the amount of time that passed between the previous and current events. Step length is the difference between the position of the foot making initial contact, and the adjusted position of the opposite foot.

Typically, each measurement is stored in memory in association with a corresponding label, which may be accomplished by ensuring that the IMU measurement devices and gold standard reference measurement are synchronized—which also yields timestamps of the temporal events which are as accurate as a few milliseconds, or better. Any suitable method may be employed to synchronize the systems (IMU measurement devices and gold standard reference measurement system), such as calibration of their internal clocks with each other or determining and adjusting for their offset if the reference system's internal clock is accessible. Or, if the internal clock is not accessible, a high-frequency video (e.g. with 100 or more frames per second) may be used to capture the internal clock presented by the IMU measurement device and the events being measured by the reference system. By detecting the first temporal event measured by the reference system in the video, the system's clock offset, which refers to the video time, may be found. By watching the device clock's timestamp, the offset with the device may be determined.

Once an ML model is available that extracts temporal events from a repetitive motion presentation, another synchronization may be performed as follows: first, the times of each left heel strike (or initial contact) from the device and the reference measurement are computed, then the cross-correlation between the timestamps of each system (IMU measurement devices and gold standard reference measurement system) are computed. Then the system may adjust the measurements to ensure that the peak of the cross-correlation occurs at time 0. This process may be done iteratively to increase the accuracy of the synchronization, and in turn the accuracy of the ML model of temporal events.

Examples of algorithmic structures of ML models to extract gait parameters e.g. gait analysis standard parameters, are now described.

a. Temporal models: A temporal model may get a repetitive motion representation, and may produce 4 (say) indices corresponding to the four temporal events. For instance, a neural network may be trained whose input layer is a 6×100 layer corresponding to the structure of the repetitive motion representation. The network's last layer may be a 4×100 layer representing 4 distributions of each of the 4 events' probabilities to occur in each time unit/index. The inner architecture of the neural network may include plural layers and plural hidden variables. The loss function for training this model may follow the KL divergence loss function to estimate the labeling distributions indicating the events' true indices with peaks.

The plural inner layers typically include a cyclic convolutional layer for processing inputs based on a repetitive motion representation. This layer is similar to a convolutional layer with padding, however, the padding is selected to make the model invariant to the starting point of the repetitive motion representation (phase 0). Convolutional layers are affected by the boundaries of the previous layer, and it is desired for the cyclic convolutional layer to equally process the data as may be rotated by any index. To accomplish this, the input data may be added, e.g. with a copy of the complementary edges on each side for the required length (which typically depends on the convolutional layer's kernel).

b. Spatial models: a spatial model's structure may be similar, but models may differ from one another in the datasets used to train the models. A spatial model may comprise a regression model which may get a repetitive motion representation and may estimate a number. For instance, a neural network may be trained whose input layer is a 6×100 layer corresponding to the structure of the repetitive motion representation. The network's last layer may be a single number layer. The inner architecture of the neural network may include plural hidden variables and plural layers including, typically, a cyclic convolutional layer e.g. as described above.

The loss function for training this model may be the mean square error to fit the labels as much as possible.

### Bodily Positions of the IMU Sensor

The process described here may be performed for the IMU device set in various bodily positions. For each position, another ground truth dataset may be gathered and a ML model may be trained against that dataset, yielding plural models for plural bodily positions.

It is appreciated that gait parameter extraction may include segmentation into repetitive patterns, each of which typically comprises a stride, or stride by each of the patient's 2 feet. One known method for segmentation of a motion record, into strides, is described in co-pending published US patent application re Assessment of a user's gait (Application #20200289027 issued Sep. 17, 2020) which describes segmentation of a motion record, into strides. Specifically, this co-pending patent document describes “Operation c-d: Segmentation into Strides” as follows: “Many methods for stride detection are known; acceleration data is typically segmented along the time axis, of into strides. Methods for performing such segmentation are described e.g. in: Perez, Andres A., and Miguel A. Labrador. “A smartphone-based system for clinical gait assessment.” 2016 IEEE International Conference on Smart Computing (SMARTCOMP). IEEE, 2016; and/or

Gadaleta, Matteo, and Michele Rossi. “Idnet: Smartp)hone-based gait recognition with convolutional neural networks.” Pattern Recognition 74 (2018): 25-37. Alternatively, rotation data may be segmented, along the time-axis, into strides. Any stride detection technique may be employed. Described herein is an example of a heuristic method to extract segments of strides from rotation data; alternatively however, the methods of Perez/Labrador or Gadaleta/Rossi may be employed.

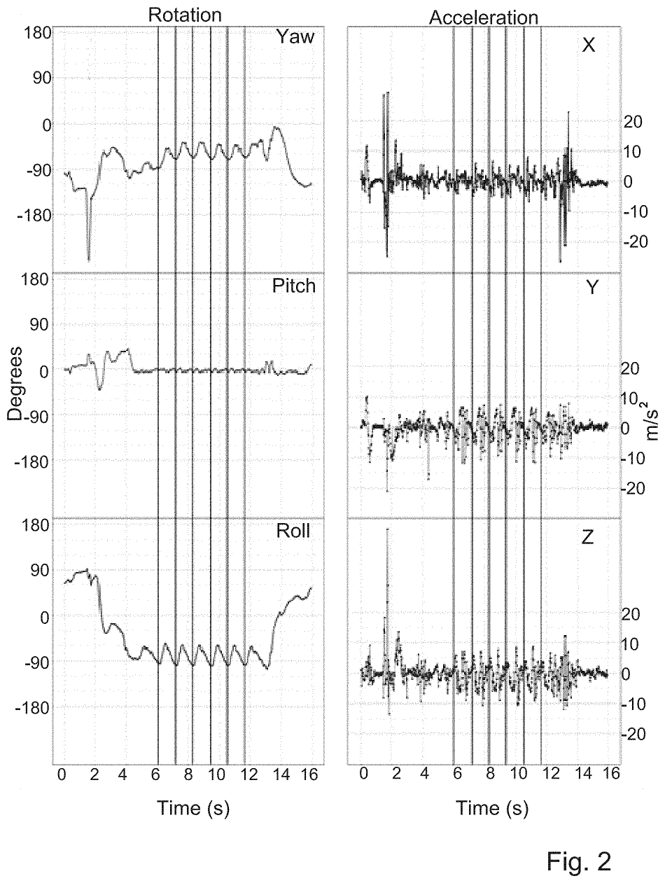

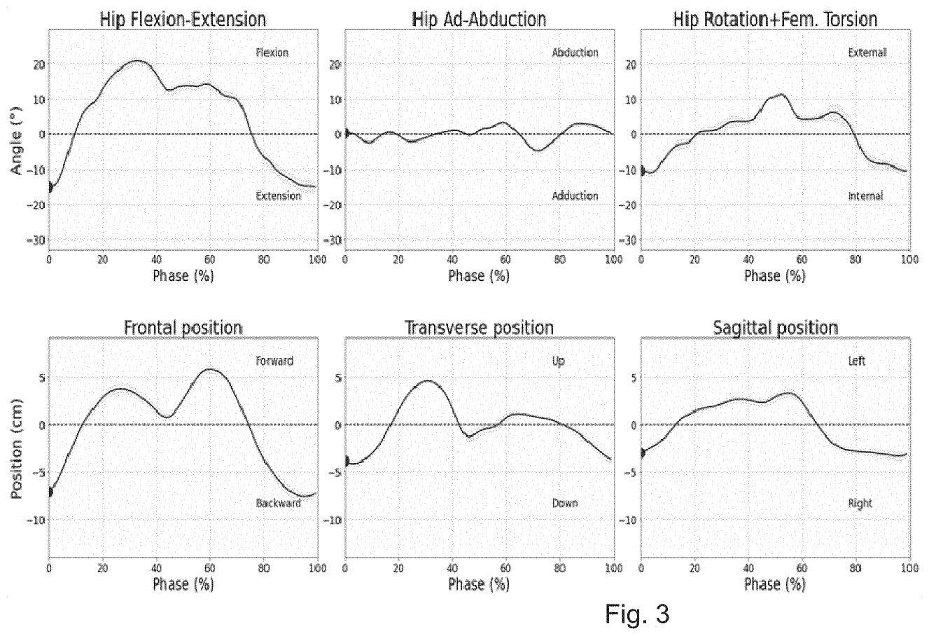

An example of stride segmentation results generated from the motion record rotation data in FIG. 2b, is shown in FIGS. 3a-3b.

Example Pseudo-code for segmentation into strides; all or any subset of lines of the following code may be used:

Let Acc ∈^{3Xn} be the acceleration data Let Rot ∈^{3Xn} be the rotation data //Check if the motion record is not static 1. velocity=cumsum(Acc, axis=1) 2. energy=sum(sum(velocity**2, axis=0), axis=1) 3. if(energy<energy_threshold): 3.1. return None //Ignore the yaw component of Rot 4. smooth_Rot=Rot=[1:,:] //Make rotation smoother with convolution, mask size is 0.25 second in sampling rate unit 5. smooth_Rot=conv(smooth_Rot, GaussianID(0.25*freq)) //Take the main component of rotation deviation 6. smooth_Rot=PCA.fit_transform(smooth_Rot)[0,:]//Finding extremum point is straight forward 7. extremum_indexes=find_extremum_points(smooth_Rot) 8. extremum_indexes_a=extremum_indexes[::2]9. extremum_indexes_b=extremum_indexes/[1::2] 10. range_a=std(smooth_Rot[extremum_indexes_a]) 11. range_b=std(smooth_Rot[extremum_indexes_b]) 12. if(range_a>range_threshold OR range_b>range_threshold) 12.1. return None 13. duration_a=std(diff(extremum_indexes_a)) 14. duration_b=std(diff(extremum_indexes_b)) 15. if(duration_a>duration_threshold OR duration_b>duration_threshold) 15.1. return None 16. strong_forces=sum(Acc**2, axis=0)<Sum forces locally (0.25 second) 17. strong_forces=conv(strong_forces, Rect(0.25*freq)) 18. sum_force_a=sum(strong_forces[extrernum_indexes_a]) 19. sum_force_b=sum(strong_forces[extremum_indexes_b]) 20. if(sum_force_a>sum_force_b) 20.1. return extremum_indexes_a 21. return extremum_indexes_b”

An example method for identification and Representation of a Repetitive Pattern Measured by IMU is now described with reference to FIGS. 1-3. Specifically, the method is configured to identify a repetitive pattern in sensor data measured by an inertial measurement unit aka “IMU) data” or to generate a standard representation of that repetitive pattern, if a repetitive pattern exists, and to utilize properties of the standard form for analysis. A repetitive pattern typically implies that while the IMU data was being generated, the IMU underwent specific repetitive motion activity e.g. because the sensor was worn by an end-user who was, as the data was being generated, engaging in walking, running, exercising, or any other movement composed of repetitions. Typically, the IMU data undergoes standardization e.g. as described herein, typically before the repetitive pattern, if any, is detected and, if detected, segmented. Reconstruction of the standard representation of the pattern, if any, typically includes.

Adjustment and Standardization of repetitions, and/or Determination of the course of movement, and/or Aggregation of the repetitions. Any suitable method may be used to provide and standardize IMU data.

The IMU may use a combination of several sensors, including all or any subset of accelerometers, gyroscopes, and sometimes magnetometers and other sensors such as a barometer, to provide, typically, at least 3 channels of spatial orientation and 3 channels of acceleration over time in various sampling rates with, typically, at least a frequency of 50 hz or higher. The IMU utilizes some of its measurements to eliminate gravitational forces from the IMUs' output, and different algorithms (such as a Kalman filter) to produce an “elementary data stream” comprising more accurate channels of orientation and acceleration (e.g. without the gravitational influence) at 50 hz at least. Additional channels such as altitude overtime may be provided, and if so, is considered extra information. An example of a data record of a 16 second time window is shown in FIG. 1.

Raw data may be standardized in terms of continuity of measurements and/or in terms of consistency of interval between measurements. The device orientation may be measured by the device's rotation in yaw, pitch, and roll, from a given direction e.g. north. Most sensors produce rotation angles in bounded range (for example, the yaw angle is between −180 to 180 degrees), so when the rotation exceeds the range, it starts over. This operation may be reversed to yield data which is continuous, as is reality. This may be seen in the upper left portion of FIG. 1; as the yaw angle drops down and exceeds the value range, the difference between the “fixed” raw data in dots, and the standardized reversed data as a continuous line, is apparent. Any suitable method may be employed to standardize (reverse the “starting over” operation), such as, for example: each time a discontinuity is detected, using some suitable definition of discontinuity such as a difference between two adjacent data points which exceeds a threshold such as, say, 180 degrees, the system may add or subtract 360 degrees to eliminate this difference, or to bring the difference between the threshold.

Motion sensors ostensibly have determined sampling rates, however it turns out that in practice, their sampling points of time are not exact. Converting the raw data into consistent intervals, i.e. a sequence of values in fixed points of time spaced equally in a specific rate, may be accomplished by interpolation of the data in time. Fortunately, a sensors' possible sampling rate is about 100 samples per second, and may thus be converted to, say, 50 samples per minute. Linear interpolation is typically good enough, e.g. as may be seen in FIG. 1 the raw data fit the standardized data very well.

### Detection and Segmentation of a Repetitive Pattern

Any suitable conventional method for segmentation of a repetitive pattern may be employed, and FIG. 2 presents the results of segmentation of a repetitive pattern over the same motion record shown in FIG. 1.

An example method described here, first identifies a repetitive pattern. The method typically comprises one or both of the following two stages:

stage 1: Sampling pattern candidates, each of which comprises a data segment. It is appreciated that it may not be known a priori, which candidate data segments actually exhibit the repetitive pattern, hence are “successful candidates”. For example, it may be desired to identify a gait cycle which is the repetitive pattern which a patient is walking. However, some data segments (IMU recordings over an interval of time e.g.) may be “unsuccessful candidates” because they turn out to have been recorded when the patient was sitting or standing, rather than walking. Also, the length of a gait cycle (pattern duration), for various subjects, may vary from 0.5 seconds to, say, 4 seconds. Thus, a segment of a given length such as 2 seconds, may represent one subject's entire gait cycle, half of a cycle for another subject, and 2 or 3 cycles for a third, fast moving patient.

Typically, the repetitive pattern is a segment (e.g. sequence) of the original data which occurs several times. Thus, finding the pattern by sampling is doable. Sampling of candidates may comprise taking data segments with a fixed duration every x seconds. In order to reflect a significant pattern, the candidates may have a long enough duration. For example, a 2-second duration may be used, sampling candidates every 4 seconds.

stage 2: Scoring the candidates with a score which reflects the objective of this process which is finding a pattern that describes the data well e.g. a pattern which, if repeated, at the indexes of repetitions, this reconstructs the original data accurately e.g. with minimal or below threshold Euclidean distance between the original data and the reconstructed data. Therefore, the better and more accurately the candidate covers the data, the more preferable that candidate is. For instance, the correlation of the candidate with the data may be computed over time over each of the channels. The total correlation over time is the mean correlation of all of the channels' correlations. Peaks of the total correlation over time are detected with standard signal processing methods, configured to filter in only peaks with the highest values over a sliding window of fixed duration. Peaks with high correlation values imply a repetition of the pattern, however the repetition may be shorter than the duration of the pattern, since correlated data intervals may overlap with one another. Hence, the real repetition is either the data interval that matches the pattern duration, or the interval between correlation matches (peaks with high values of correlation), typically the shorter of the two. Eventually, the score may be computed as a sum of the repetition durations multiplied by their correlation values which may be regarded as reflecting the measure of coverage that the pattern candidate captures. A candidate is dismissed if no sequential repetitions are detected, indicating that this pattern is not repetitive. Parameters configured for this process may include: peaks' sliding window duration parameter which may be determined by the shortest repetition expected; e.g. the parameter may be 0.4 seconds as capturing a significant pattern with higher frequency than this value enables is not expected. Other parameters configured for this process may include the correlation threshold to consider a peak as a potential repetition, and/or the minimal number of consecutive repetitions, and/or the maximum deviation of the intervals' durations between consecutive repetitions. Example parameters' values may be: correlation threshold: 0.5, minimal number of consecutive repetitions: 3, maximum deviation of the intervals' durations between consecutive repetitions: 40% of deviation from the average duration.

Once the candidate with the highest score has been identified, the following stages should be carried out:
1. Refinement of the pattern—the segments between consecutive repetitions have the correct length of the repetitive pattern; moreover, their average pattern may be shown to achieve a higher average correlation with all the repetitions. So the average of the segments between repetitions would get a higher score as a candidate, and therefore, may be deemed the “true” repetitive pattern. All segments may be cut to fit the shortest interval between repetitions, to avoid overlaps.
2. Detection of the repetitions—find peaks of the total correlation between the final pattern and the original data, e.g. as was done when evaluating the scores of the pattern candidates; the same configured parameters as before may be used again. Only intervals between consecutive peaks as repetitions may be considered; although this might miss the first and the last repetitions of a sequence or sequences of two repetitions, this ensures that the considered intervals are real instances (repetitions of) the repetitive pattern. In addition, the first and the last repetitions in a sequence might be a bit different than the rest of the repetitions. Thus, typically, precise detection of real repetitions is prioritized over “covering” all repetitions (over the goal of detecting every single repetition).

### Reconstruction of the Pattern Representation

The measurement may be represented in a reference frame defined as follows: the X-axis is the course of the movement of the IMU (of the end-user wearing the IMU e.g.), the Y-axis is, say, toward the sky, the Z-axis is, say, toward the right (the normal of the X-Y plane). The following operations yield a representation of the repetitions which typically captures all relevant information of the motion being measured while being invariant to irrelevant attributes of the measurement and the analysis, including the base orientation at which the device was set during the measurement, and phase 0 at which the repetitions define the starting point of the repetitive pattern. Once the following 3 stages have been performed, the output comprises a standard representation of the pattern, including 3 spatial channels, 3 orientational channels, and a channel for each extra information channel of the original data, with over 100 uniform units dividing the duration of the repetitions

Stage a—Adjustment and Standardization of Repetitions

For each of the data segments of the repetitions, perform all or any subset of the following operations, suitably ordered e.g. as shown:
1. Ignoring the offset of the yaw channel—The north is irrelevant to defining the frame of reference as defined above, and similarly any other offset of the yaw, since given a motion record of someone walking, the direction of the walk is not relevant, and it is desirable for the representation of that activity to be invariant of the direction. Hence, the average yaw may be subtracted for each repetition, whether the data is calibrated to the north, or showing a false reference of the north. Other techniques may also be used, such as splitting the yaw channel into two—one channel for the azimuth (a smoothed version of the yaw represents the macro changes of the yaw), and the other channel for subtraction of the azimuth from the yaw, which represents the micro changes of the yaw.
2. The uncalibrated reference frame—Although at this stage the directions of the X- and Z-axis are not known, the Y-axis (the vertical direction) is known from the orientation channel of the data. Applying (e.g. using conventional rotation arithmetic) the rotations included in the orientation channel, the acceleration channel takes these rotations from the reference frame of the measurement device and embeds these rotations in an “uncalibrated” reference frame”, e.g. a reference frame in which the X or the Z axes are arbitrary; they may be found as described in stage B as described below. Multiplying the rotations of the orientation channel with their inverse mean of orientation on the right produces rotations that are relative to the mean orientation of the segment, aka the relative rotations. By rotating the acceleration channel and producing the relative rotations, the relation between the data and the orientation of the measurement system over the body is eliminated, and what remains is accelerations and relative rotations in the uncalibrated reference frame.
3. Displacement and orientation adjustment—typically, the data is presented as displacement, although the data could also be presented as accelerations. The displacement is smoother, enables understanding of the motion better for human eyes, and preserves the information of the original data, which may be reversed by derivating the displacement. The reference frame moves with the movement, meaning that the displacement over the data segment may be assumed to be 0 which determines the initial velocity (the velocity may also be assumed not to change over the data segment, meaning that there are no accelerations between repetition, which turns out to be a reasonable assumption for long continuous repetitions. In addition, the effect on the data is minor, which is subtraction of the average acceleration over the data segment (the average acceleration may be kept). This assumption yields benefits: the data may be fixed due to noise or biases of the sensors which are reasonable, and the displacement is not only closed (as it starts and ends at the same displacement) but also smooth, since the velocity is the same at the beginning and at the end. This makes any shift of data from the end of the segment to its beginning yield the same displacement, yielding invariance to the determination of phase 0, the starting point of the repetitions' segments. The orientation of the starting point and the last point may be constrained to be the same.
   Thus, according to certain embodiments, the average acceleration is subtracted from the accelerations and integrated t over the segment to get velocities; then the average velocity is subtracted from the velocities and integrated over the segment to get the displacement. Orientations are handled after the reference frame has been fixed.
4. Interpolation—interpolate the displacement and the orientation of each segment to have (say) 100 equal time units, to ensure a standard structure for each repetition.
   Stage b—Determination of the Course of Movement

Rotate the reference frame such that X and Z axes will be calibrated, where the course of movement is the X-axis. This rotation depends on the orientation of the measurement system referred to the motion itself; typically, all of the repetitions' representations at this stage are embedded in coherent reference frames, which typically need to be calibrated with the same rotation. This rotation may be estimated using any suitable technique, such as applying principal component analysis (PCA) on the projections of either the displacements or the orientations on the horizontal plane (which is known, since the vertical axis is known). The most significant component of the displacement may imply the direction of the movement because this is the direction in which the most significant changes typically occur. Similarly, the most significant component of the orientations represented as rotation vectors may imply the Z-axis around which the most significant changes of orientations typically occur (e.g. in walking measured from the thigh (e.g. if the IMU is adjacent to the end-user's thigh), the most significant component of the orientations over the horizontal plane is the hip flexion-extension axis). Either of these (the most significant component of the displacement, or the most significant changes of orientations) may be used to rotate the reference frame. Eventually, this yields all of the data represented in a calibrated frame of reference. The orientations may then be converted to Euler representation in the order of Z-X-Y, and the average angle velocities of each orientation channel may be subtracted to verify each of them starts and ends at the same angle, which yields invariance to the initial point of the segmentations e.g. as described above.

Stage c—Aggregation of the Repetitions

The repetitions' representations are similar to each other, even initially, and, typically, after standardization of their structure and elimination of irrelevant attributes of measurement and analysis, the repetitions' representations become even more similar. Some tasks may use one aggregated representation of the motion, rather than on each of the repetitions, for example, recognition of the activity being performed by the subject and the position of the measurement device as well. For this kind of task, the aggregated form of the repetitions' representations may be employed. This process may, for example, comprise naive averaging each of the channels over every one of 100 (say) time-units or by dynamic time warping (DTW) techniques. In practice, the naive aggregation method, while less sophisticated, nonetheless works well.

It is appreciated that, alternatively, the data segmentations may be taken as-is, and stages a, b, c may be omitted.

The representation shown and described above is useful for a broad variety of tasks, such as but not limited to any or all of the following:
1. Activity recognition—given a representation of repetitive motion—the task of classifying the activity performed by the subject and/or classifying the position of the sensor e.g. IMU on the body, as the activity was performed and measured.
2. Gait analysis—producing standard clinical characteristics of walking, such as temporal parameters describing durations and symmetry of phases such as stance and swing of the two legs, and spatial parameters such as each legs' step length and velocity.
3. Exercising evaluation or assigning scores or levels to exercise quality parameters—repetitive motion exercises such as measurement of range of motion for some joints, stairs climbing, squats, and other exercises, include repetitions of movement. Evaluation may involve recording all or any subset of the following exercise quality parameters: a number of repetitions performed by the patient or end-user, their rate, the range of different angles or range of motion, and other characteristics for each specific exercise, such as, for a squatting exercise, how long a patient manages to stay in the squatting position, how fast or slowly the patient then gets up, and the angle of the patient's thigh in her or his lowest position.
4. Motion capture—producing body joints kinematics to demonstrate how the patient aka subject walks e.g. a partial set of joints such as only lower body joints, or full-body joints.

Referring again to stage a, it is appreciated that the orientational or angular channel—as described above, is useful for measuring exercise quality e.g. in measuring the angle achieved by a subject's arm in an arm waving exercise, or between the subject's upper and lower leg, in a squat exercise.

For an exercise such as the above, determination of the course of movement may include, rather than applying a PCA on the horizontal plane to find the course of the walk, applying the PCA on the 3D spatial vector representation of the orientations, and thus finding the general most significant component of the change of orientation which may be used as a value for the angle of the squat or arm-wave.

Example methods for Extraction of Exercise Quality Parameters from a Single IMU Data of an Exercise Recording are now described in detail with reference to FIGS. 4-6.

The methods are configured to extract exercise quality parameters from an exercise measured by an inertial measurement unit (IMU), and typically make use of all or any subset of the following functionalities:
1. Generating a representation of a repetitive motion pattern measured by an IMU—e.g. as described herein with reference to FIGS. 1-3.
2. Recognition of type of activity (e.g. walking, ascending stairs, descending stairs, running, squatting, pedaling a bicycle etc.) and bodily position (e.g. of the sensor, relative to the subject's body; for example, was the sensor in the patient's hip pocket) of a representation of a repetitive motion—e.g. as described herein in the co-pending gait assessment patent document referred to elsewherein herein (published U.S. Ser. No. 16/659,832); and
3. Gait parameter extraction methods e.g. as described herein.

As opposed to spontaneous movement, an exercise is structured, and hence its movement is expected. An exercise may include a clinical standard test which includes a structured movement.

An exercise may include plural repetitions, in which case a sensed representation of the exercise includes a repetitive pattern. Exercise quality parameters may describe a single repetition of an exercise with plural repetitions, or may describe the exercise as a whole.

The extraction of parameters describing a single repetition of exercise may follow a scheme of a machine learning (ML) framework, and may include:
1. Collecting ground truth data for training and validation of parameter extraction; and/or
2. Outlining the general structure of models for parameter extraction
   Thus Exercise Quality Parameters may include all or any subset of:
1. Exercise Quality Parameters based on repetitions, which are derived from the repetitive motion representation directly
2. “ML parameters”: Exercise Quality Parameters based on repetitions, which are extracted using machine learning aka ML
3. Exercise Quality Parameters that evaluate the exercise as a whole

### Repetition Based Exercise Quality Parameters

These are extracted by algorithms that do not require a training (fitting) process and may include all or any subset of the following:
Duration [seconds]: The duration of repetitions may be derived from the repetitive motion representation directly.
Cadence [repetition/min]: number of repetitions per minute, inferred from the cycle durations.
Range of (angular) motion [degrees]: the range of most significant change of orientation.

When calibrating the reference frame of the repetitive motion representation, taking the most significant component in PCA analysis of the orientation change, as opposed to taking the most significant component of the projection on the horizontal plane, is an adjustment more appropriate for exercise analysis. Eventually, the first angular channel represents the most significant angular change. The range of motion may be interpreted differently for different exercises, for example, how long a patient manages to stay in the squatting position while s/he performs squats, how fast or slowly the patient then gets up, and the angle of her/his thigh in her or his lowest position.

ML repetition based Exercise Quality parameters

The following are examples (all or any subset of which may be provided) of Exercise Quality parameters extracted by algorithms that employ an ML process.
Spatio-temporal gait parameters: e.g. as described in the context of gait parameter extraction methods described herein.
Height of jumps [cm]: produced similarly to the spatial gait parameters described in the context of gait parameter extraction methods described herein.
The reference measurement may be a video camera synced with the IMU internal timestamp (e.g. by capturing the clock with the camera). After hundreds (say) of subjects are measured, manual annotation may be used to label each jump and produce ground truth for training a spatial parameter model e.g. as described herein, or using any conventional method.
Execution quality of a specific exercise: Evaluation of execution quality of a specific exercise (and detection of activity, and detection of position, may be based on the aggregated representation of the repetitive motion. This evaluation functionality may be configured to predict the modification or approval that a human expert would give as feedback for the execution of that exercise. Typically, the labels are not required to be synchronized accurately. Ground truth may be provided as follows: hundreds (say) of subjects are filmed performing the exercise, and the exercise is also sensed by an IMU system positioned in the subjects' pants pockets. A human expert divides the performance according to the videos, into categories of feedbacks. For example, squats execution would be human classified into the following categories: performed well, back was bent, knees went inwards. In this case, this task employs a classification model rather than a regression model. A neural network model trained for this task ends with a layer which may include neurons, each of which indicates the probability of each category for the motion representation input. Training that model over the ground truth typically follows the cross-entropy loss function.

Exercise Quality Parameters describing an entire exercise may include assessments of exercise e.g.:

Timed up and go duration [seconds]: “Timed up and go” is a standard clinical test in which a seated patient is instructed to (“forward” stage) stand up, walk 10 meters forward, turn around, and then (in the “return” stage), to go back to sit on the chair. The duration of performing this test has a clinical interpretation of the patient's condition. A measurement of performing this standard test may look as presented in FIG. 4 in which the motion is segmented into sitting intervals, standing up, sitting down, and a walking interval. The walking “window” includes initial and terminal movements (shakes), straight strides, and turning. The segmentation may be produced as follows: first, the gait strides are detected e.g. as described herein with reference to FIGS. 1-3. As explained in stage B of the reconstruction of the repetitive pattern representation, in walking measured from the thigh (e.g. if the IMU is adjacent to the end-user's thigh e.g. is in the subject's hip pocket), the most significant component of the orientations over the horizontal plane is the hip flexion-extension axis, which is the first orientational channel. Also, the sitting intervals are detected, as their acceleration data indicates no motion is taking place—the average amplitude of the accelerations in a sliding window is under a threshold. Also, the standing up and sitting down intervals are segmented from the static segments until the first (significant) local maxima of the flexion occurs. The azimuth channel is typically split, at the beginning of the process, from the rest of the motion data, and may be used to validate that the movement is back and forth, and/or to indicate where the turning is. Eventually, the system may verify that the pattern is a gait pattern e.g. that the activity the subject is engaged in, is walking. Any suitable method may be used to recognize the activity a subject is engaged in, e.g. by analysis of a representation of a repetitive motion e.g. as described in co-pending published U.S. Ser. No. 16/659,832, and may estimate the distance using the stride length using any suitable gait parameter extraction method to extract the stride length, such as those described elsewhere herein.

If all the details are valid for, or are consistent with, a “timed up and go test” (i.e. 10 meters of walking, back and forth), the system may conclude that this measurement is a valid test, and may return the duration from standing up to sitting down.

Typically, an “activity” is repetitive, hence comprises a sequence of repetitive patterns. For example, walking is an activity, which comprises a sequence of gait cycles.

FIG. 4 is a graph showing an example of flexion and acceleration amplitude extracted from IMU data. The flexion is the first angular (orientation) channel produced by the process e.g. as described herein. The calibration rotation may be applied over the entire IMU data, rather than to the individual pattern repetitions only. The activity recorded in FIGS. 4 and 5 show the forward and return stages, respectively, of a patient or subject performing a timed up and go test.

Another example of an exercise quality parameter, is the number of seconds of Stability that a subject may achieve, while standing on one leg.

In order to verify the position of the phone and the capability of the measurement to assess a quality parameter of a stability test, the user may be instructed to place the phone in his pocket, and take several steps. This enables the position of the phone to be determined, e.g. as described in previously published co-pending patent documents e.g. the co-pending gait-assessment patent document. FIG. 6 presents a measurement of such an exercise. Specifically, FIG. 6 shows flexion and acceleration amplitude extracted from IMU data recorded while a subject, with a phone in his pocket, was standing in one leg. The flexion is the first angular channel produced by the process e.g. as described herein, where the calibration rotation is applied over the entire IMU data, rather than over individual pattern repetitions only. The activity recorded in FIG. 6 shows a user performing a stability exercise while standing on one leg. The measurement is taken from the pants pocket of the leg raised in the air. The segmentation of the repetitions at the beginning of the graph shows gait cycles, followed by a swing of the leg and a stabilization interval, and then a static interval which corresponds to the stable leg being held in the air. In the edges of the recording, segments of transitions are present, indicating a change in the position of the measurement device. Detection of static intervals may be produced as described in above in the context of “timed up and go”. Transitions may be identified where no repetitive motion is detected, and nor is a static interval, and, in addition, the average orientation of the device, before and after the segment, changes significantly (e.g. changes to an over-threshold extent). The exercise quality parameter may measure the duration of the first static interval that occurs after stabilization. Once the raised leg starts to shake, the measurement is ended.

It is appreciated that the system herein provides an infrastructure for testing hypotheses regarding various groups or profiles of similar patients. An example hypothesis is: does a smaller number of exercises yield better adherence?

It is appreciated that parameters used by any of the systems or methods herein may include derived parameters as opposed to or in addition to raw sensor data. Some parameters may be “actively recorded” e.g. via an app serving the patient and/or therapist, and may be used to distinguish between intentional activity and unconscious activity, and some may be generated from an “always on monitor” e.g. as described in the following co-pending application (https://patents.iustia.com/patent/20200289027. Individual patient parameters may subdivide into demographic parameters and gait parameters, vs. patient-group parameters vs. exercise parameters (difficulty, length of time per repetition, etc.) vs. exercise execution parameters subdividing into adherence, compliance) vs. treatment plan (aka exercise plan) parameters (e.g. how many minutes per day). Gait parameters may be subdivided into gait during therapy and daily life gait (outside of therapy).

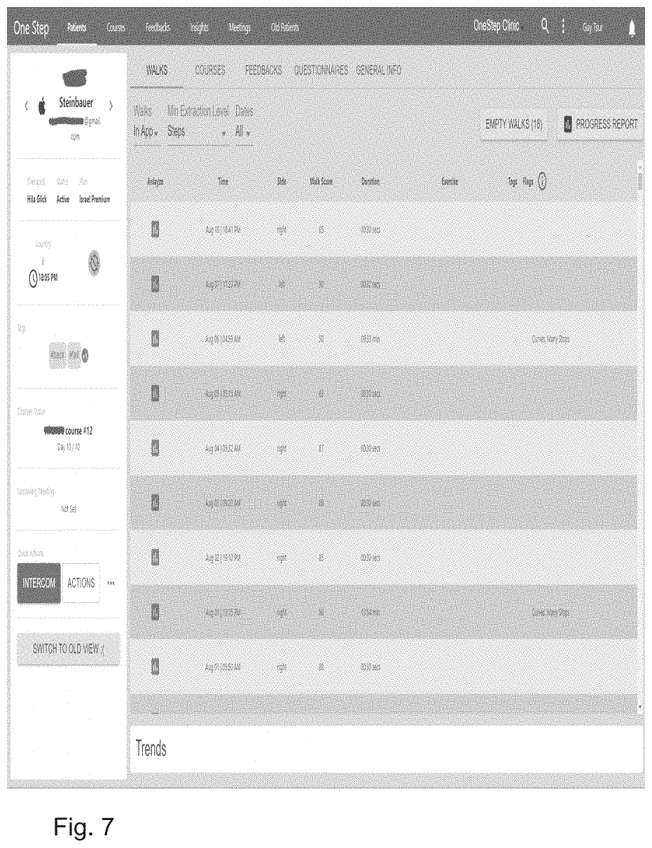

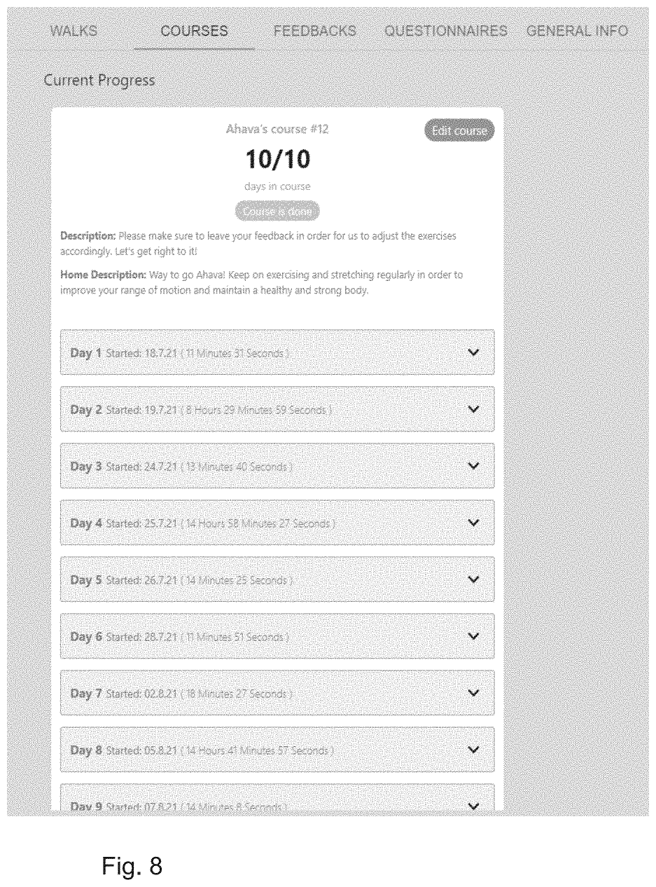

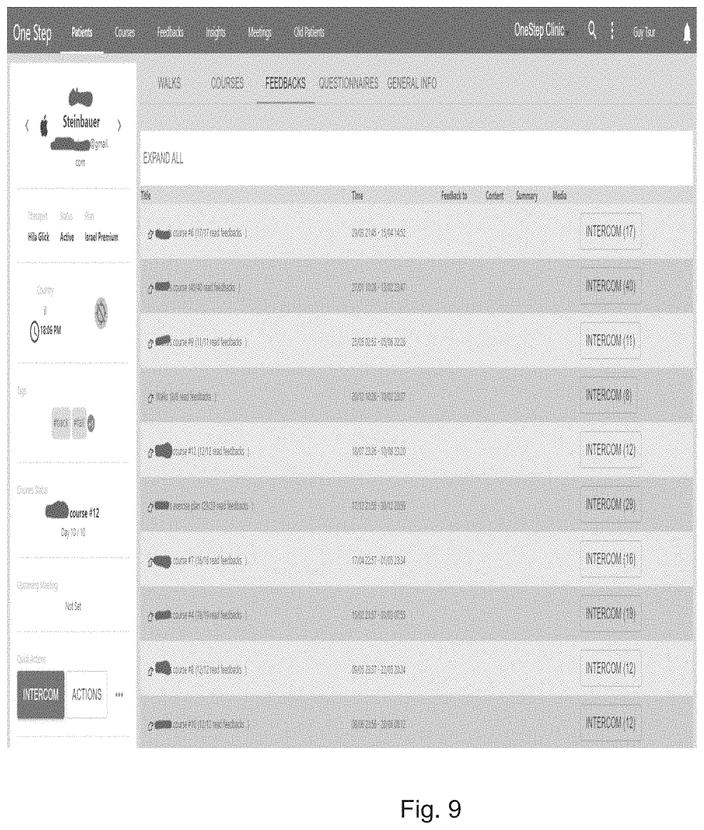

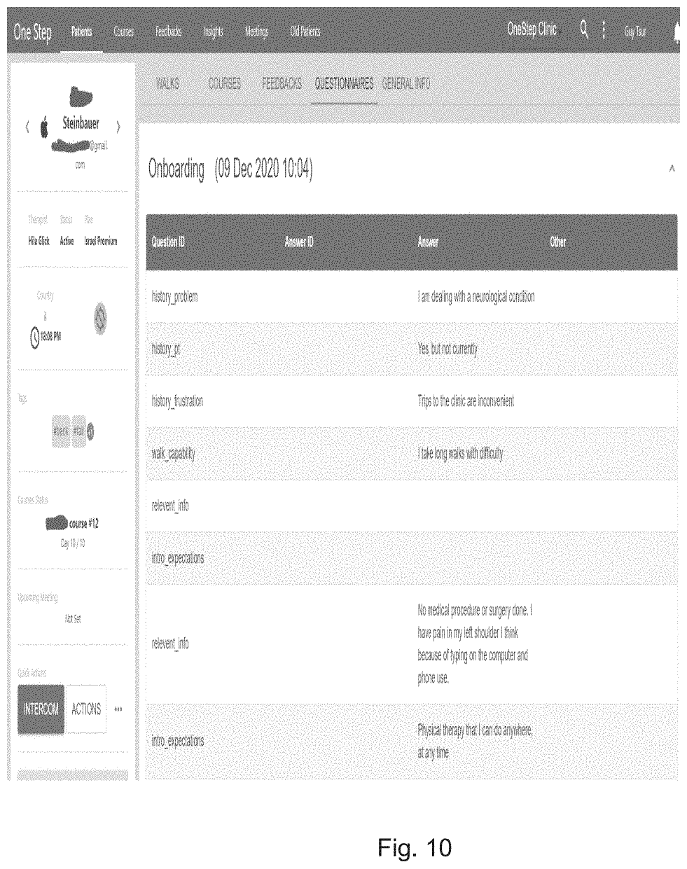

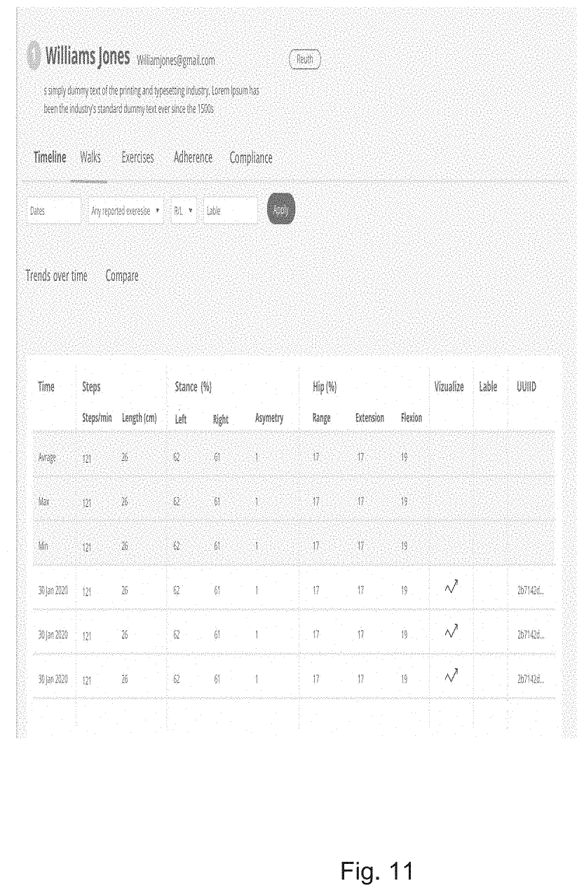

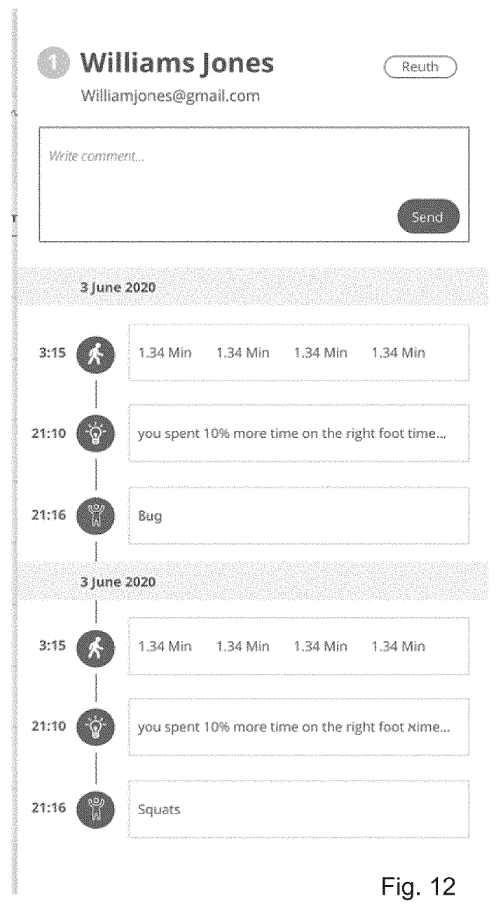

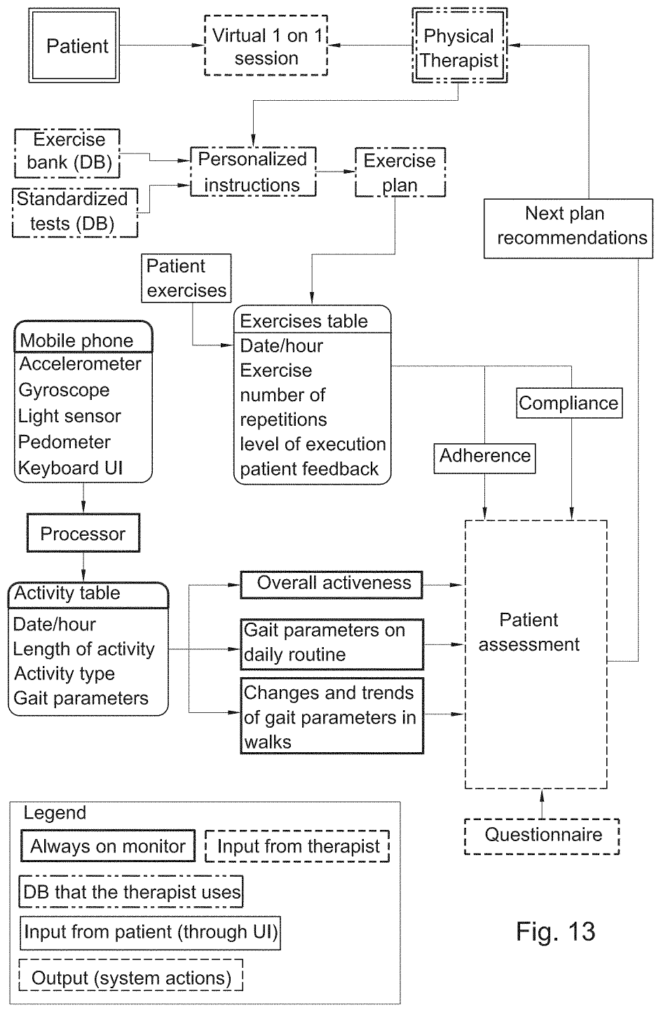

The app may include any suitable GUI. For example, FIGS. 7-12 are simplified pictorial illustrations of an example GUI, wherein all or any subset of FIGS. 7-12, and of the elements illustrated in each, may be provided e.g. as a dashboard for PTs or other human experts, and/or for patient end-users. The app may have any suitable architecture e.g. as shown by way of example in the simplified block diagram of FIG. 13.

It is appreciated that references herein to regression, are not intended to be limiting. For example, If the data is multivariate, multivariate analysis, rather than regression, may of course be used.

Also, according to certain embodiments, regression may be applied over all patients in a training data set (e.g. all patients in a specific subgroup e.g. the post-stroke subgroup or the males-over-age-40 subgroup, or all patients in all subgroups), to get coefficients. The coefficients may then be used to predict improvement of patients in test data set. Any suitable AI technique, including but not limited to machine learning, may then be employed to compare predicted improvements of patients in test data set to actual improvements of patients in test data set, and then learn even better regression coefficients for subsequent regression analyses.

According to certain embodiments, the system is configured to estimate improvement using adherence and compliance data. The system may estimate various improvement parameters e.g. the system may be configured to find (or fit or learn) a function that inputs the adherence and the compliance of patients (in a subgroup, over various different exercises) and returns a score correlated with the improvement. Example: given a period of time, the improvement achieved over that period of time is known. The system may then compute, for each exercise type, its adherence frequency per week (e.g. once a week) and the final level of compliance (e.g. if the final level of compliance >70% that exercise is highly successful=a score of 1, if the final level of compliance >50% that exercise is fairly successful=a score of 0.5, and otherwise, the exercise's score is poor=0). At least one vector may be stored in the system, wherein each index or component of the vector, corresponds to an exercise. Each patient with a period of known improvement is represented as a vector, and the values of the vector are the multiplication of the frequency with the level of compliance. The system may fit a linear regression over different patients' vectors, where the dependent variable of the regression is defined as the improvement score of the relevant patient. The contribution of an exercise is the component weight corresponding to the exercise index (e.g. unique identifier of a given exercise).

Alternatively, assume for simplicity that the contribution of each patient's exercise is independent of contributions of other patients and/or of other exercises. Hence, each exercise for each patient in a period of improvement has the improvement over the period, and its adherence and compliance. This yields, for any subset of patients, a set of many tuples, each including adherence and compliance as independent variables and improvement as the dependent variable Y. The system may then learn the relation between the X (adherence, compliance) and the Y (improvement) for an exercise, for a subset of patients. For example, if high adherence and compliance levels of an exercise correlate with significant improvement, the system may conclude that this exercise is recommended for this group of patients.

It is appreciated that providing representations of a repetitive pattern identified in (typically wearable) sensor data such as IMU data has a wide variety of use-cases such as but not limited to the uses specifically shown and described herein. Thus use cases include but are not limited to gait analysis e.g. on the go or while an end-user is wearing the sensor or in real time or in near-real time, motion capture systems which may be based on a single device, or more than one, which may be worn by the moving object e.g. human e.g. may measure from the moving object's pocket/s, with or without recommendation functionality such as the exercise recommendation functionality shown and described herein.

The scope of the present invention includes any system or method which provides gait analysis, according to any embodiment herein, and/or exercise analysis, according to any embodiment herein, and/or recommendation functionality, according to any embodiment herein, which generates a recommendation (such as an exercise recommendation) for an end-user (professional and/or patient/subject/layman), derived from at least one output of the gait analysis and/or of the exercise analysis. Alternatively or in addition, the system or method may present e.g. display to an end-user (professional and/or patient/subject/layman) at least one output of the gait analysis and/or of the exercise analysis. The gait analysis and/or exercise analysis may be derived from data provided by on an IMU or sensor/s deployed in a single (e.g. off the shelf, standard) cellular phone which may be worn by the subject or patient or end-user whose gait or exercising is being monitored. Or, sensors may be deployed in more than one bodily position on the end-user's body. A particular advantage of certain embodiments is that a subject's gait may be analyzed throughout the day; the subject may not even need to be aware of this e.g. may not need to activate or de-activate or otherwise interact with the functionality, according to certain embodiments.

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required, or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform e.g. in software any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone, or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g. by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g. web-based system employing software, computers, routers and telecommunications equipment, as appropriate.

Any suitable deployment may be employed to provide functionalities e.g. software functionalities shown and described herein. For example, a server may store certain applications, for download to clients, which are executed at the client side, the server side serving only as a storehouse. Any or all functionalities, e.g. software functionalities shown and described herein, may be deployed in a cloud environment. Clients, e.g. mobile communication devices such as smartphones, may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if -then” logic described herein is intended to include embodiments in which a processor is programmed to repeatedly determine whether condition x, which is sometimes true and sometimes false, is currently true or false and to perform y each time x is determined to be true, thereby to yield a processor which performs y at least once, typically on an “if and only if” basis e.g. triggered only by determinations that x is true, and never by determinations that x is false.

Any determination of a state or condition described herein, and/or other data generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may, for example, comprise changing the state or condition, or may more generally cause any outcome which is technically advantageous given the state or condition or data, and/or may prevent at least one outcome which is disadvantageous given the state or condition or data. Alternatively or in addition, an alert may be provided to an appropriate human operator or to an appropriate external system.

Features of the present invention, including operations, which are described in the context of separate embodiments may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art, and particularly, although not limited to, those described in the Background section, or in publications mentioned therein.

Conversely, features of the invention, including operations, which are described for brevity in the context of a single embodiment, or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation and is not intended to be limiting.

Any suitable communication may be employed between separate units herein e.g. wired data communication and/or in short-range radio communication with sensors such as cameras e.g. via WiFi, Bluetooth or Zigbee.

It is appreciated that implementation via a cellular app as described herein is but an example, and, instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.), or may be conventionally tethered to a networked device (to a device which is a node in a conventional communication network, or is tethered directly or indirectly/ultimately to such a node).

Any operation or characteristic described herein may be performed by another actor outside the scope of the patent application, and the description is intended to include apparatus whether hardware, firmware or software which is configured to perform, enable or facilitate that operation, or to enable, facilitate or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry including any such computer microprocessor/s as well as in firmware or in hardware or any combination thereof.

It is appreciated that elements illustrated in more than one drawing, and/or elements in the written description, may still be combined into a single embodiment, except if otherwise specifically clarified herewithin. Any of the systems shown and described herein may be used to implement or may be combined with, any of the operations or methods shown and described herein.

It is appreciated that any features, properties, logic, modules, blocks, operations or functionalities described herein which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately or in any suitable sub-combination, including with features known in the art. Each element e.g. operation described herein, may have all characteristics and attributes described or illustrated herein, or, according to other embodiments, may have any subset of the characteristics or attributes described herein.

It is appreciated that apps referred to herein may include a cell app, mobile app, computer app or any other application software. Any application may be bundled with a computer and its system software, or published separately. The term “phone” and similar used herein is not intended to be limiting and may be replaced or augmented by any device having a processor, such as but not limited to a mobile telephone, or also set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node). Thus, the computing device may even be disconnected from e.g., WiFi, Bluetooth, etc. but may be tethered directly or ultimately to a networked device.

1. A sensor-driven physiotherapy system comprising:
an exercise repository electronically storing digitally represented exercises each of which includes at least one exercise quality parameter;
a hardware processor configured to assign a score for at least one exercise quality parameter for each of at least some exercises in the repository of exercises, wherein the score is derived from sensor data collected while patients performed said exercises wearing a sensor which generates said sensor data; and
exercise recommendation functionality configured to select, from said digitally represented exercises, at least one exercise which the system recommends for at least one profile of patient, wherein the exercise which the system recommends, for a given profile of patient, statistically contributes more, to gait improvement of patients belonging to said given profile, than exercises which are not recommended for said given profile contribute, to gait improvement of patients belonging to said given profile.

2. A system according to claim 1 wherein the hardware processor includes gait parameter estimation logic which estimates at least one gait parameter from sensor data.

3. A system according to claim 1 wherein said at least one exercise, which the system recommends for at least one profile of patient, includes first and second exercises, wherein the first exercise statistically contributes more to gait improvement, of patients belonging to said given profile, than the second exercise, and wherein the first exercise is displayed to end-users of the system before the second exercise is displayed.

4. A system according to claim 1 wherein said hardware processor's ability to derive said score from said sensor data is machine learned.

5. A system according to claim 2 wherein the system also comprises an app used by patients during physiotherapy exercise sessions, and wherein at least one individual patient's gait is estimated by analyzing sensor data collected at times when the individual patient is not interacting with the app.

6. A system according to claim 2 wherein the at least one gait parameter is estimated by the hardware processor, from sensor data pertaining to only one single gait cycle having a gait cycle duration.

7. A system according to claim 6 wherein the at least one gait parameter is estimated by extraction of predefined gait events for each leg and determining proportions e.g. percentages of the gait cycle duration which are occupied by said predefined gait events respectively.

8. A system according to claim 6 wherein the gait events include at least one of heel-strike for right leg, heel-strike for left leg, toe off for right leg, and toe-off for left leg.

9. A system according to claim 7 wherein said extraction is machine-learned.

10. A system according to claim 9 wherein said extraction which is machine-learned receives a repetitive motion representation, comprising a standard representation of a repetitive motion identified in said sensor data, and uses a temporal machine-learning model to produce indices respectively corresponding to the gait events.

11. A system according to claim 10 wherein the standard representation of the repetitive motion is fed to a classifier which classifies which activity is being performed by the patient.

12. A system according to claim 10 wherein the standard representation of the repetitive motion is fed to a classifier which classifies the bodily position at which the sensor is deployed.

13. A system according to claim 10 wherein, to identify said repetitive motion in said sensor data, the sensor data is standardized by providing continuity of measurements generated by the sensor.

14. A system according to claim 10 wherein, to identify said repetitive motion in said sensor data, the sensor data is standardized by providing consistency of intervals between measurements generated by the sensor.

15. A system according to claim 10 wherein the repetitive motion representation has 6 components including yaw, pitch and roll components of a patient's gait's rotation and x, y and z components of a patient's gait's acceleration where x is the patient's gait's direction of movement
and wherein the extraction uses a trained neural network whose input layer comprises 6 L neurons and whose last layer comprises GL neurons where G is a number of gait events to be estimated
and wherein L is a desired number of temporal subdivisions of said repetitive motion's duration thereby to yield L time-units
and wherein distributions of each of the G events' probabilities to occur in each time unit/index are provided.

16. A system according to claim 15 and wherein the trained neural network has inner layers including a cyclic convolutional layer.

17. A system according to claim 2 wherein sensor data is also collected at times other than during physiotherapy exercise sessions and is used by the hardware processor to estimate at least one gait parameter G at times other than during physiotherapy exercise sessions and wherein said gait parameter G is used to quantify said gait improvement.

18. A system according to claim 2 wherein the at least one gait parameter include at least one of: step length, stride length, stride width.

19. A system according to claim 2 wherein the at least one gait parameter includes at least one temporal clinical standard parameter.

20. A system according to claim 2 wherein the at least one gait parameter includes at least one spatial clinical standard parameter.

21. A system according to claim 7 wherein when said extraction is machine-learned, and machine learning is performed plural times for each of plural bodily positions in which the sensor may be deployed, thereby to yield plural machine learning models trained against plural respective ground truth datasets.

22. A system according to claim 2 wherein the hardware processor is configured to estimate improvement of a patient's gait, by deriving said improvement from said at least one gait parameter extracted from said sensor data.

23. A system according to claim 1 wherein said sensor comprises an IMU.

24. A system according to claim 1 which provides an output indication of body joint kinematics for at least one joint of at least one patient, thereby to present a visualization of the patient's gait.

25. A system according to claim 1 wherein said exercise quality parameters include at least one of the following non-machine learned exercise quality parameters: a number of repetitions performed by the patient, a cadence parameter, and an indication of a change of the patient's orientation.

26. A computer program product, comprising a non-transitory tangible computer readable medium having computer readable program code embodied therein, said computer readable program code adapted to be executed to implement a sensor-driven physiotherapy method comprising:
storing digitally represented exercises each of which includes at least one exercise quality parameter, thereby to provide an exercise repository;
using a hardware processor to assign a score at least one exercise quality parameter for each of at least some exercises in the repository of exercises, wherein the score is derived from sensor data collected while patients performed said exercises wearing a sensor which generates said sensor data; and
selecting, from said digitally represented exercises, at least one exercise which the system recommends for at least one profile of patient, wherein the exercise which the system recommends, for a given profile of patient, statistically contributes more to gait improvement of patients belonging to said given profile, than exercises which are not recommended for said given profile contribute, to gait improvement of patients belonging to said given profile.

27. A sensor-driven physiotherapy method comprising:
storing digitally represented exercises each of which includes at least one exercise quality parameter, thereby to provide an exercise repository;
using a hardware processor to assign a score at least one exercise quality parameter for each of at least some exercises in the repository of exercises, wherein the score is derived from sensor data collected while patients performed said exercises wearing a sensor which generates said sensor data; and
selecting, from said digitally represented exercises, at least one exercise which the system recommends for at least one profile of patient, wherein the exercise which the system recommends, for a given profile of patient, statistically contributes more to gait improvement of patients belonging to said given profile, than exercises which are not recommended for said given profile contribute, to gait improvement of patients belonging to said given profile.
